(19) United States
(12) Patent Application Publication
(10) Pub. No.: US 2024/0217973 A1
(43) Pub. Date: Jul. 4, 2024

(54) IMIDAZOPYRIDINE COMPOUNDS AND USE THEREOF

(71) Applicant: MEDSHINE DISCOVERY INC., Jiangsu (CN)

(72) Inventors: Yang ZHANG, Shanghai (CN); Wentao WU, Shanghai (CN); Kaijun GENG, Shanghai (CN); Rongxin HUANG, Shanghai (CN); Shuhui CHEN, Shanghai (CN)

(21) Appl. No.: 18/554,644

(22) PCT Filed: Apr. 15, 2022
(86) PCT No.: PCT/CN2022/087228

§ 371 Date: Oct. 10, 2023

(30) Foreign Application Priority Data
Apr. 16, 2021 (CN) 202110413879.2
Mar. 2, 2022 (CN) 202210200324.4

(51) Int. Cl. C07D 471/04; A61K 31/437; A61K 31/4545; A61P 37/00

(52) U.S. Cl. CPC C07D 471/04; A61K 31/437; A61K 31/4545; A61P 37/00

(57) ABSTRACT

Provided are a series of imidazopyridine compounds and a use thereof, and in particular, relating to compounds as represented by formula (P), and pharmaceutically acceptable salts thereof.

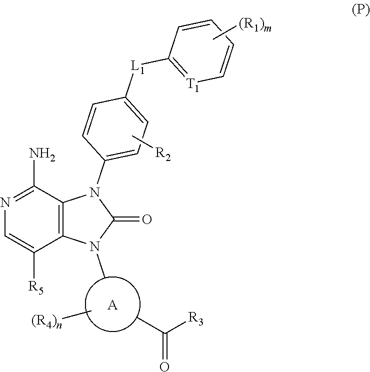

The present application claims the right of the following priorities: CN202110413879.2, application date: Apr. 16, 2021; CN202210200324.4, application date: Mar. 2, 2022.

### TECHNICAL FIELD

The present disclosure relates to a series of imidazopyridine compounds and a use thereof, in particular to a compound as represented by formula (P) and a pharmaceutically acceptable salt thereof.

### BACKGROUND

Bruton's tyrosine kinase (BTK) is a member of the Tec family (TEC family kinases, TFKs) of tyrosine kinases, which consists of five members, in addition to BTK, there are ITK, TEC, BMX and TXK. BTK is expressed in B cells, macrophages and monocytes, but not in T cells. BTK plays a crucial role in signal transduction through the B cell receptor (BCR) and Fc γ receptors in B cells and myeloid cells.

BTK is mainly responsible for the transmission and amplification of various intracellular and extracellular signals in B lymphocytes, which is necessary for B cells to mature. Inactivation of BTK function in XLA patients can lead to a deficiency of peripheral B cells and immunoglobulins. The signal receptors upstream of BTK include growth factor and cytokine receptors, G protein coupled receptors such as chemokine receptors, antigen receptors (especially B cell receptors [BCR]) and integrins. BTK in turn activates many major downstream signaling pathways, including phosphoinositide-3 kinase (PI3K)-AKT pathway, phospholipase-C (PLC), protein kinase C, and nuclear factor κB (NF-κB), and so on. The role of BTK in BCR signal transduction and cell migration has been well established, and these functions seem to be the main targets of BTK inhibitors. The increase of BTK activity was detected in blood cancer cells such as B-cell chronic lymphoblastic leukemia (CLL) and mantle cell lymphoma (MCL). Abnormal activity of BTK function often leads to B cell malignancies or autoimmune diseases, making it a popular research and development target.

The BTK inhibitors currently approved by the FDA include ibrutinib, acalabrutinib and zanubrutinib. The main indications include mantle cell lymphoma, Waldenström's macroglobulinemia, small lymphocytic lymphoma and marginal zone lymphom, which are also popular targets in the field of immune diseases such as rheumatoid arthritis and systemic lupus erythematosus.

### CONTENT OF THE PRESENT INVENTION

The imidazopyridine compounds of the present invention are a type of protein kinase inhibitor, which has a variety of therapeutic applications and can be used to treat proliferation, inflammation, autoimmunity and other related disorders caused by protein kinases. The present disclosure provides a series of imidazopyridine-based irreversible BTK inhibitors.

The present disclosure provides a compound represented by formula (P) or a pharmaceutically acceptable salt thereof,

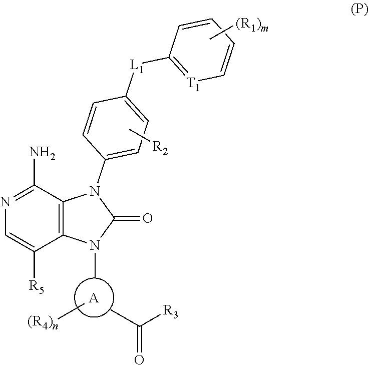

wherein
L₁ is selected from O and —C₁₋₃ alkyl-NH—C(═O)—;
ring A is selected from pyrrolidinyl and piperidyl;
each R₁ is independently selected from halogen, C₁₋₃ alkyl and C₁₋₃ alkoxy, respectively, and the C₁₋₃ alkyl and C₁₋₃ alkoxy are independently optionally substituted by 1, 2 or 3 halogens, respectively;
R₂ is selected from H, halogen and C₁₋₃ alkyl, and the C₁₋₃ alkyl is optionally substituted by 1, 2 or 3 halogens;
R₃ is selected from

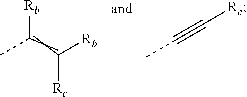

R₄ is selected from halogen, CN, C₁₋₃ alkyl and C₁₋₃ alkoxy, and the C₁₋₃ alkyl and C₁₋₃ alkoxy are independently optionally substituted by 1, 2 or 3 halogens, respectively;
R₅ is selected from H, halogen and C₁₋₃ alkyl, and the C₁₋₃ alkyl is optionally substituted by 1, 2 or 3 halogens;
each R_{b} is independently selected from H and halogen, respectively;
each R_{c} is selected from H and C₁₋₃ alkyl, respectively, and the C₁₋₃ alkyl is substituted by 1, 2 or 3 F;
m is 0, 1, 2 or 3;
n is 0, 1 or 2;
provided that when Li is selected from O, R_{c} is selected from C₁₋₃ alkyl, and the C₁₋₃ alkyl is substituted by 1, 2 or 3 F.

In some embodiments of the present disclosure, L₁ is selected from O and —CH₂—NH—C(—O)—, and other variables are as defined in the present disclosure.

In some embodiments of the present disclosure, each R₁ is independently selected from F, Cl, Br, CH₃, CH₂CH₃, CH(CH₃)₂, OCH₃, OCH₂CH₃ and OCH(CH₃)₂, respectively, and the CH₃, CH₂CH₃, CH(CH₃)₂, OCH₃, OCH₂CH₃ and OCH(CH₃)₂ are independently optionally substituted by 1, 2 or 3 halogens, respectively, and other variables are as defined in the present disclosure.

In some embodiments of the present disclosure, each R₁ is independently selected from F, Cl, Br, CH₃, CH₂CH₃, CH(CH₃)₂, CF₃, OCH₃, OCH₂CH₃, OCH(CH₃)₂ and OCF₃, respectively, and other variables are as defined in the present disclosure.

In some embodiments of the present disclosure, the structural moiety

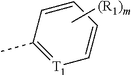

is selected from

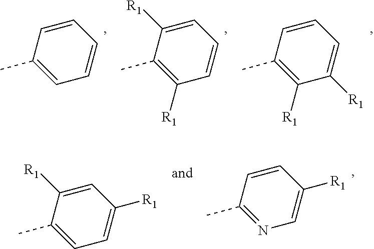

and other variables are as defined in the present disclosure.

In some embodiments of the present disclosure, the structural moiety

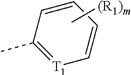

is selected from

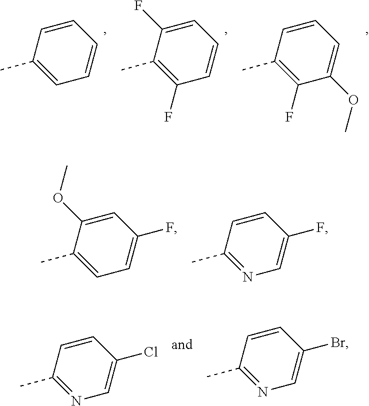

and other variables are as defined in the present disclosure.

In some embodiments of the present disclosure, R₂ is selected from H, F, Cl and CH₃, and other variables are as defined in the present disclosure.

In some embodiments of the present disclosure, the structural moiety

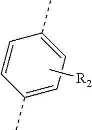

is selected from

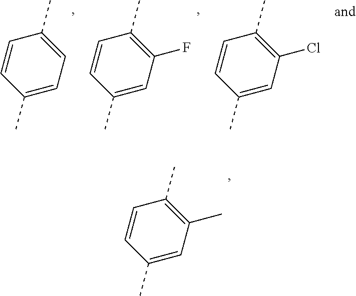

and other variables are as defined in the present disclosure.

In some embodiments of the present disclosure, each R_{c} is independently selected from H, CH₃, CH₂CH₃ and CH(CH₃)₂, respectively, and the CH₃, CH₂CH₃ and CH(CH₃)₂ are substituted by 1, 2 or 3 F, and other variables are as defined in the present disclosure.

In some embodiments of the present disclosure, each R_{c} is independently selected from H, CH₂F, CHF₂ and CF₃, respectively, and other variables are as defined in the present disclosure.

In some embodiments of the present disclosure, R₃ is selected from

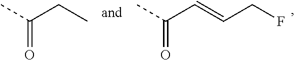

and other variables are as defined in the present disclosure.

In some embodiments of the present disclosure, R₄ is selected from F, Cl, Br, CN, CH₃, CF₃, OCH₃ and OCF₃, and other variables are as defined in the present disclosure.

In some embodiments of the present disclosure, R₄ is selected from F and CH₃, and other variables are as defined in the present disclosure.

In some embodiments of the present disclosure, the ring A is selected from

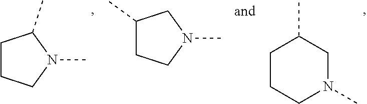

and other variables are as defined in the present disclosure.

In some embodiments of the present disclosure, n is 0 or 1, and other variables are as defined in the present disclosure.

In some embodiments of the present disclosure, the structural moiety

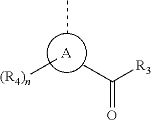

is selected from

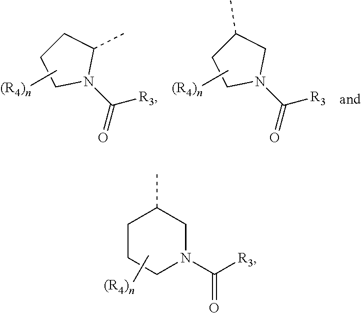

and other variables are as defined in the present disclosure.

In some embodiments of the present disclosure, the structural moiety

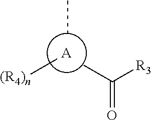

is selected from

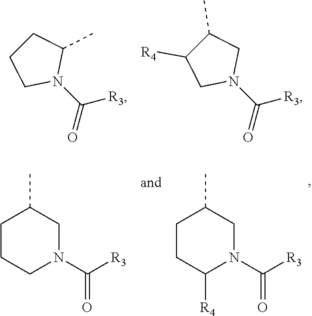

and other variables are as defined in the present disclosure.

In some embodiments of the present disclosure, the structural moiety

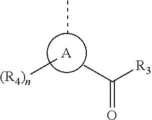

is selected from

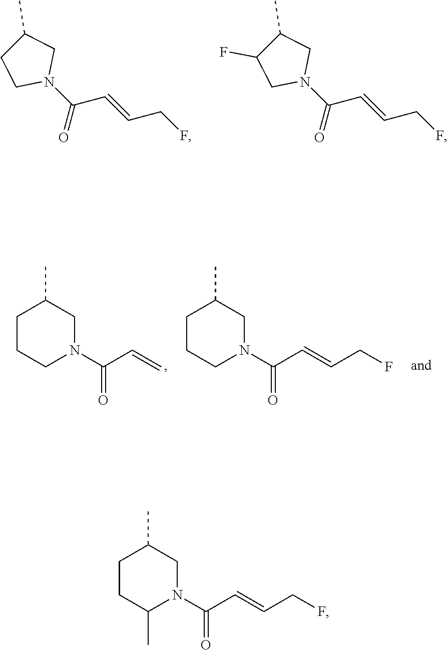

and other variables are as defined in the present disclosure.

In some embodiments of the present disclosure, R₅ is selected from H, and other variables are as defined in the present disclosure.

The present disclosure provides a compound represented by formula (II) or a pharmaceutically acceptable salt thereof,

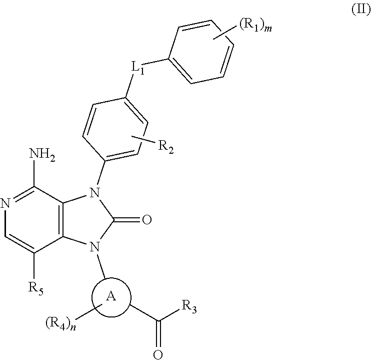

wherein
L₁ is selected from O and —C₁₋₃ alkyl-NH—C(═O)—;
ring A is selected from pyrrolidinyl or piperidyl;
each R₁ is independently selected from halogen, C₁₋₃ alkyl and C₁₋₃ alkoxy, respectively, and the C₁₋₃ alkyl and C₁₋₃ alkoxy are independently optionally substituted by 1, 2 or 3 halogens, respectively;
R₂ is selected from H, halogen and C₁₋₃ alkyl, and the C₁₋₃ alkyl is optionally substituted by 1, 2, or 3 halogens;
R₃ is selected from

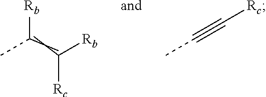

R₄ is selected from halogen, CN, C₁₋₃ alkyl and C₁₋₃ alkoxy, and the C₁₋₃ alkyl and C₁₋₃ alkoxy are independently optionally substituted by 1, 2 or 3 halogens, respectively;
R₅ is selected from H, halogen and C₁₋₃ alkyl, and the C₁₋₃ alkyl is optionally substituted by 1, 2 or 3 halogens;
each R_{b} is independently selected from H and halogen, respectively;
each R_{c} is selected from H and C₁₋₃ alkyl, respectively, and the C₁₋₃ alkyl is substituted by 1, 2 or 3 F;
m is 0, 1, 2 or 3;
n is 0, 1 or 2;
provided that when Li is selected from O, R_{c} is selected from C₁₋₃ alkyl, and the C₁₋₃ alkyl is substituted by 1, 2 or 3 F.

In some embodiments of the present disclosure, L₁ is selected from O and —CH₂—NH—C(═O)—, and other variables are as defined in the present disclosure.

In some embodiments of the present disclosure, each R₁ is independently selected from F, Cl, Br, CH₃, CH₂CH₃, CH(CH₃)₂, OCH₃, OCH₂CH₃ and OCH(CH₃)₂, respectively, and the CH₃, CH₂CH₃, CH(CH₃)₂, OCH₃, OCH₂CH₃ and OCH(CH₃)₂ are independently optionally substituted by 1, 2 or 3 halogens, respectively, and other variables are as defined in the present disclosure.

In some embodiments of the present disclosure, each R₁ is independently selected from F, Cl, Br, CH₃, CH₂CH₃, CH(CH₃)₂, CF₃, OCH₃, OCH₂CH₃, OCH(CH₃)₂ and OCF₃, respectively, and other variables are as defined in the present disclosure.

In some embodiments of the present disclosure, the structural moiety

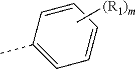

is selected from

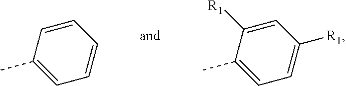

and other variables are as defined in the present disclosure.

In some embodiments of the present disclosure, the structural moiety

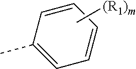

is selected from

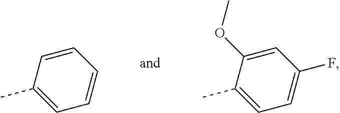

and other variables are as defined in the present disclosure.

In some embodiments of the present disclosure, R₂ is selected from H, F, CI, Br, CH₃, CH₂CH₃ and CH(CH₃)₂, and the CH₃, CH₂CH₃ and CH(CH₃)₂ are independently optionally substituted by 1, 2 or 3 halogens, respectively, and other variables are as defined in the present disclosure.

In some embodiments of the present disclosure, R₂ is selected from H, and other variables are as defined in the present disclosure.

In some embodiments of the present disclosure, each R_{c} is independently selected from H, CH₃, CH₂CH₃ and CH(CH₃)₂, respectively, and the CH₃, CH₂CH₃ and CH(CH₃)₂ are substituted by 1, 2 or 3 F, and other variables are as defined in the present disclosure.

In some embodiments of the present disclosure, each R_{c} is independently selected from H, CH₂F, CHF₂ and CF₃, respectively, and other variables are as defined in the present disclosure.

In some embodiments of the present disclosure, R₃ is selected from

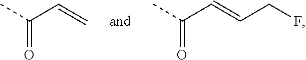

and other variables are as defined in the present disclosure.

In some embodiments of the present disclosure, the ring A is selected from

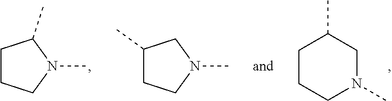

and other variables are as defined in the present disclosure.

In some embodiments of the present disclosure, the structural moiety

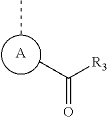

is selected from

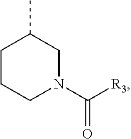

and other variables are as defined in the present disclosure.

In some embodiments of the present disclosure, the structural moiety

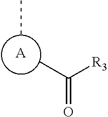

is selected from

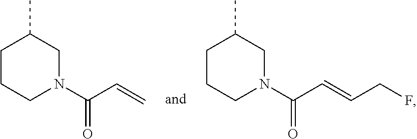

and other variables are as defined in the present disclosure.

In some embodiments of the present disclosure, R₄ is selected from F, Cl, Br, CN, CH₃, CF₃, OCH₃, and OCF₃, and other variables are as defined in the present disclosure.

In some embodiments of the present disclosure, n is 0, and other variables are as defined in the present disclosure.

In some embodiments of the present disclosure, R₅ is selected from H, and other variables are as defined in the present disclosure.

The present disclosure provides a compound represented by formula (I) or a pharmaceutically acceptable salt thereof,

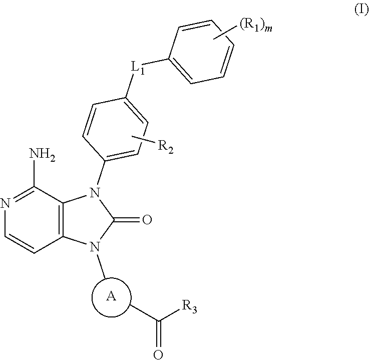

wherein
L₁ is selected from O and —C₁₋₃ alkyl-NH—C(═O)—;
ring A is selected from pyrrolidinyl or piperidyl;
each R₁ is independently selected from F, Cl, Br, C₁₋₃ alkyl and C₁₋₃ alkoxy, respectively, and the C₁₋₃ alkyl and C₁₋₃ alkoxy are optionally substituted by 1, 2 or 3 Rₐ;
R₂ is selected from H, F, Cl and Br;
R₃ is selected from

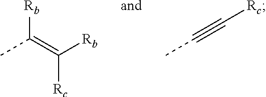

each Rₐ is independently selected from F, Cl, and Br, respectively;
each R_{b} is independently selected from H, F, Cl, and Br, respectively;
each R_{c} is selected from H and C₁₋₃ alkyl, respectively, and the C₁₋₃ alkyl is substituted by 1, 2, or 3 F;
m is 0, 1, 2, or 3;
provided that when L₁ is selected from O, R_{c} is selected from C₁₋₃ alkyl, and the C₁₋₃ alkyl is substituted by 1, 2 or 3 F.

In some embodiments of the present disclosure, L₁ is selected from O and —CH₂—NH—C(═O)—, and other variables are as defined in the present disclosure.

In some embodiments of the present disclosure, each R₁ is independently selected from F, Cl, Br, CH₃, CH₂CH₃, CH(CH₃)₂, OCH₃, OCH₂CH₃ and OCH(CH₃)₂, respectively, and the CH₃, CH₂CH₃, CH(CH₃)₂, OCH₃, OCH₂CH₃ and OCH(CH₃)₂ are optionally substituted by 1, 2 or 3 Rₐ, and other variables are as defined in the present disclosure.

In some embodiments of the present disclosure, each R₁ is independently selected from F, Cl, Br, CH₃, CH₂CH₃, CH(CH₃)₂, CF₃, OCH₃, OCH₂CH₃, OCH(CH₃)₂ and OCF₃, respectively, and other variables are as defined in the present disclosure.

In some embodiments of the present disclosure, the structural moiety

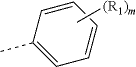

is selected from

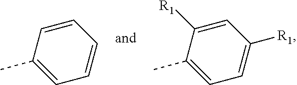

and other variables are as defined in the present disclosure.

In some embodiments of the present disclosure, the structural moiety

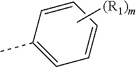

is selected from

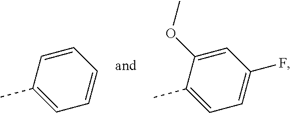

and other variables are as defined in the present disclosure.

In some embodiments of the present disclosure, R₂ is selected from H, and other variables are as defined in the present disclosure.

In some embodiments of the present disclosure, each R_{c} is independently selected from H, CH₃, CH₂CH₃ and CH(CH₃)₂, respectively, and the CH₃, CH₂CH₃ and CH(CH₃)₂ are substituted by 1, 2, or 3 F, and other variables are as defined in the present disclosure.

In some embodiments of the present disclosure, each Re is independently selected from H, CH₂F, CHF₂ and CF₃, respectively, and other variables are as defined in the present disclosure.

In some embodiments of the present disclosure, R₃ is selected from

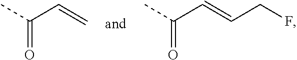

and other variables are as defined in the present disclosure.

In some embodiments of the present disclosure, the ring A is selected from

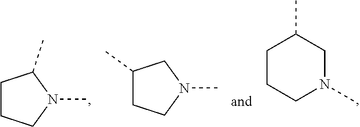

and other variables are as defined in the present disclosure.

In some embodiments of the present disclosure, the structural moiety

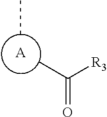

is selected from

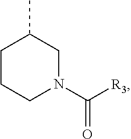

and other variables are as defined in the present disclosure.

In some embodiments of the present disclosure, the structural moiety

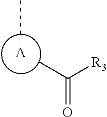

is selected from

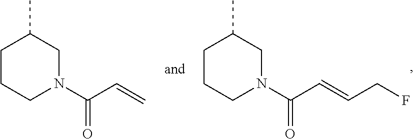

and other variables are as defined in the present disclosure.

In some embodiments of the present disclosure, the compound or the pharmaceutically acceptable salt thereof, and the compound is selected from,

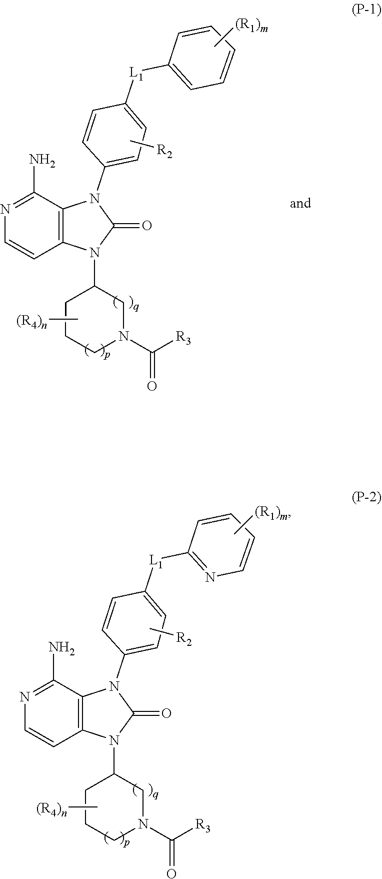

wherein L₁, R₁, R₂, R₃, R₄, m and n are as defined in the present disclosure;
p is 0 or 1, q is 0 or 1, and p and q are not 0 at the same time.

In some embodiments of the present disclosure, the compound or the pharmaceutically acceptable salt thereof, and the compound is selected from,

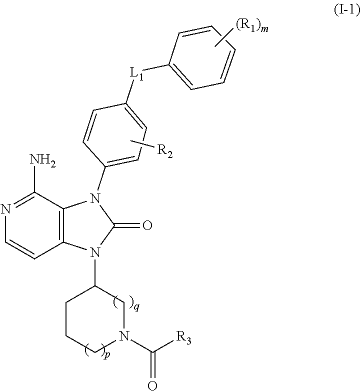

wherein L₁, R₁, R₂, R₃ and m are as defined in the present disclosure;
p is 0 or 1, q is 0 or 1, and p and q are not 0 at the same time.

In some embodiments of the present disclosure, the compound or the pharmaceutically acceptable salt thereof, and the compound is selected from,

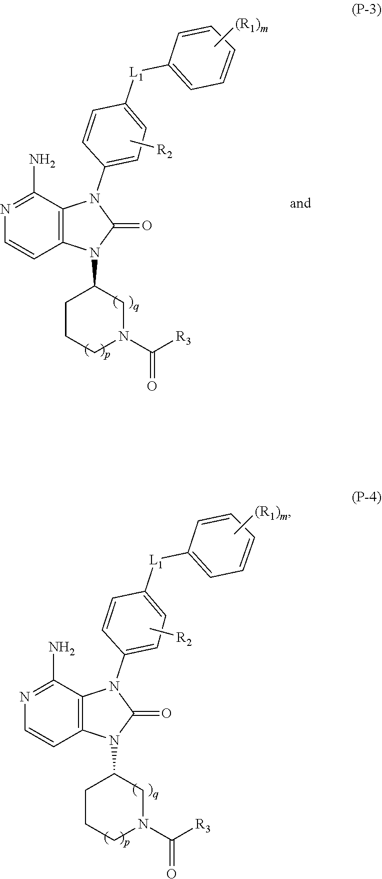

wherein L₁, R₁, R₂, R₃ and m are as defined in the present disclosure;
p is 0 or 1, q is 0 or 1, and p and q are not 0 at the same time.

There are still some embodiments of the present disclosure which are obtained by any combination of the above variables.

The present disclosure also provides a compound represented by the following formula or a pharmaceutically acceptable salt thereof,

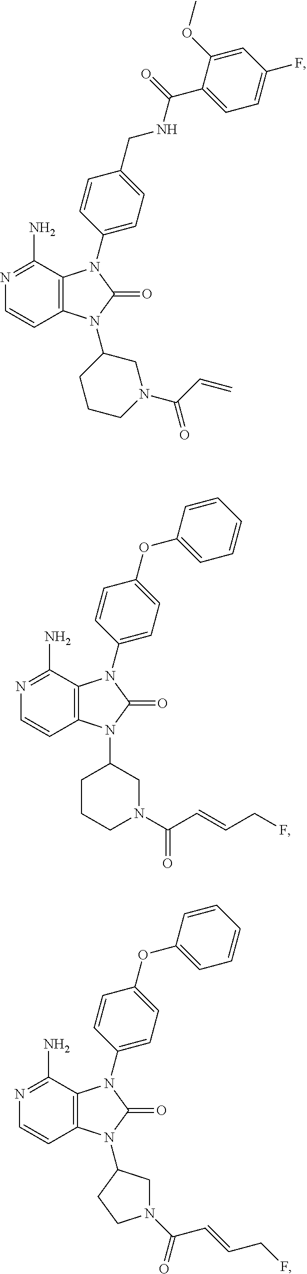 [SMILES: C=CC(=O)N1CCCC(n2c(=O)n(-c3ccc(CNC(=O)c4ccc(F)cc4OC)cc3)c3c(N)nccc32)C1 ; Nc1nccc2c1n(-c1ccc(Oc3ccccc3)cc1)c(=O)n2C1CCCN(C(=O)/C=C/CF)C1 ; Nc1nccc2c1n(-c1ccc(Oc3ccccc3)cc1)c(=O)n2C1CCN(C(=O)/C=C/CF)C1]

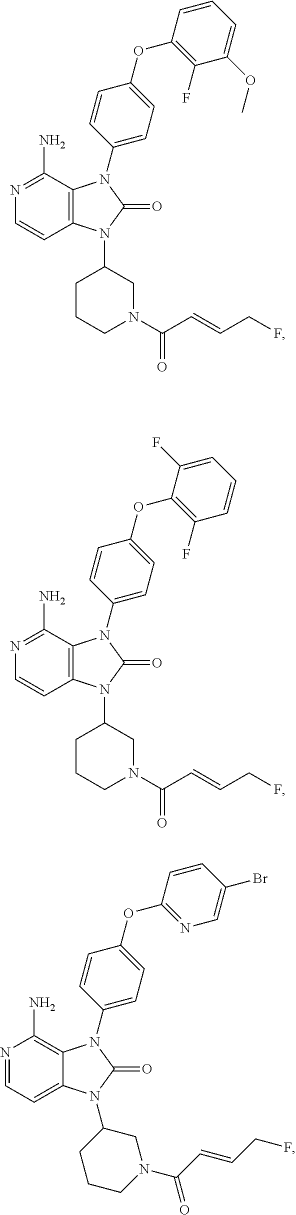 [SMILES: COc1cccc(Oc2ccc(-n3c(=O)n(C4CCCN(C(=O)/C=C/CF)C4)c4ccnc(N)c43)cc2)c1F ; Nc1nccc2c1n(-c1ccc(Oc3c(F)cccc3F)cc1)c(=O)n2C1CCCN(C(=O)/C=C/CF)C1 ; Nc1nccc2c1n(-c1ccc(Oc3ccc(Br)cn3)cc1)c(=O)n2C1CCCN(C(=O)/C=C/CF)C1]

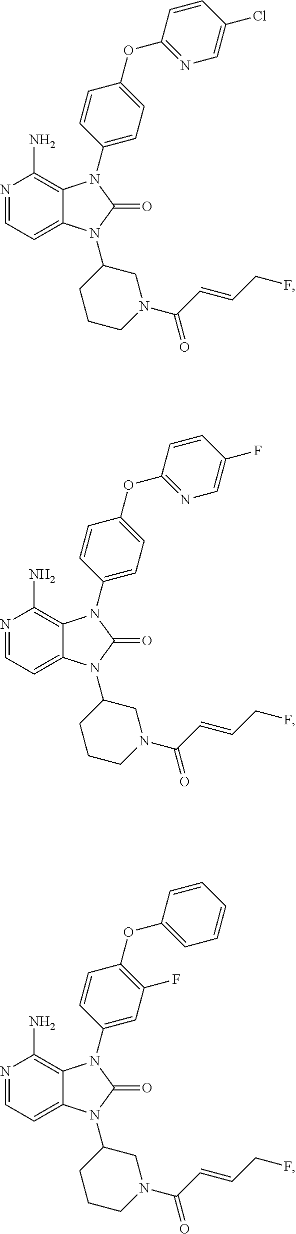 [SMILES: Nc1nccc2c1n(-c1ccc(Oc3ccc(Cl)cn3)cc1)c(=O)n2C1CCCN(C(=O)/C=C/CF)C1 ; Nc1nccc2c1n(-c1ccc(Oc3ccc(F)cn3)cc1)c(=O)n2C1CCCN(C(=O)/C=C/CF)C1 ; Nc1nccc2c1n(-c1ccc(Oc3ccccc3)c(F)c1)c(=O)n2C1CCCN(C(=O)/C=C/CF)C1]

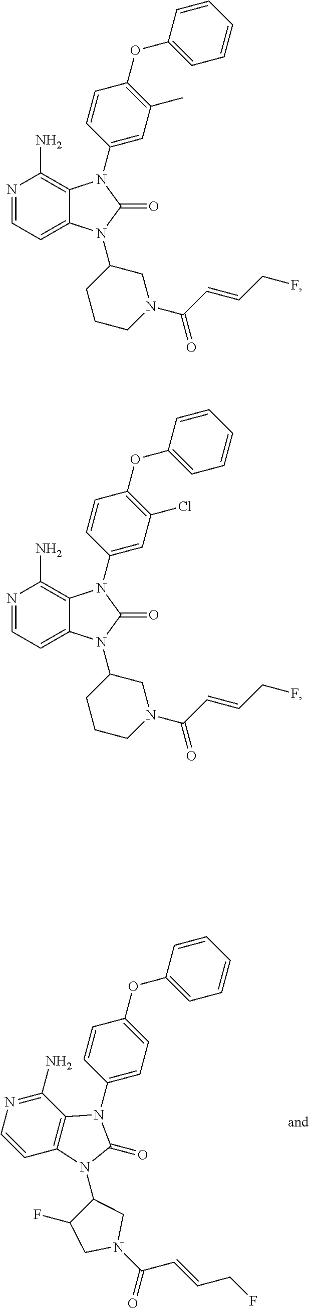 [SMILES: Cc1cc(-n2c(=O)n(C3CCCN(C(=O)/C=C/CF)C3)c3ccnc(N)c32)ccc1Oc1ccccc1 ; Nc1nccc2c1n(-c1ccc(Oc3ccccc3)c(Cl)c1)c(=O)n2C1CCCN(C(=O)/C=C/CF)C1 ; Nc1nccc2c1n(-c1ccc(Oc3ccccc3)cc1)c(=O)n2C1CN(C(=O)/C=C/CF)CC1F]

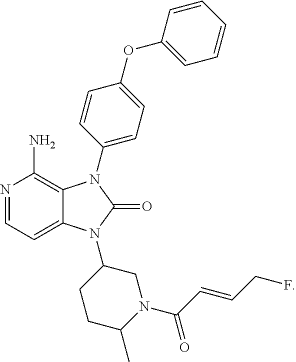 [SMILES: CC1CCC(n2c(=O)n(-c3ccc(Oc4ccccc4)cc3)c3c(N)nccc32)CN1C(=O)/C=C/CF]

In some embodiments of the present disclosure, the compound or the pharmaceutically acceptable salt thereof, and the compound is selected from,

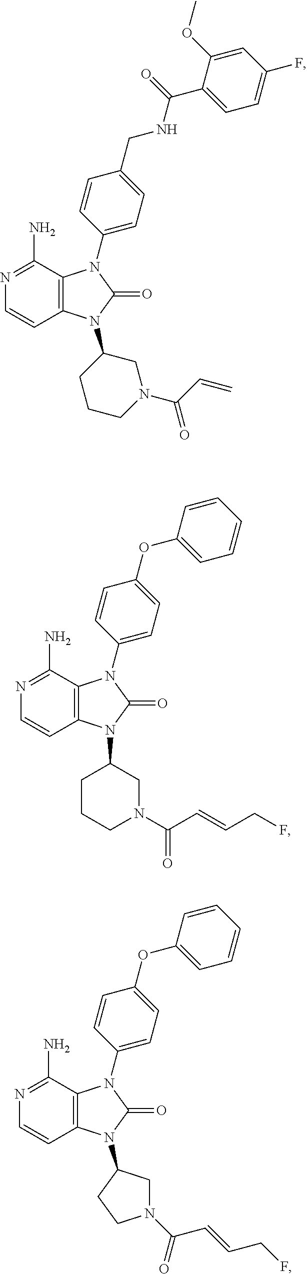 [SMILES: C=CC(=O)N1CCC[C@@H](n2c(=O)n(-c3ccc(CNC(=O)c4ccc(F)cc4OC)cc3)c3c(N)nccc32)C1 ; Nc1nccc2c1n(-c1ccc(Oc3ccccc3)cc1)c(=O)n2[C@@H]1CCCN(C(=O)/C=C/CF)C1 ; Nc1nccc2c1n(-c1ccc(Oc3ccccc3)cc1)c(=O)n2[C@@H]1CCN(C(=O)/C=C/CF)C1]

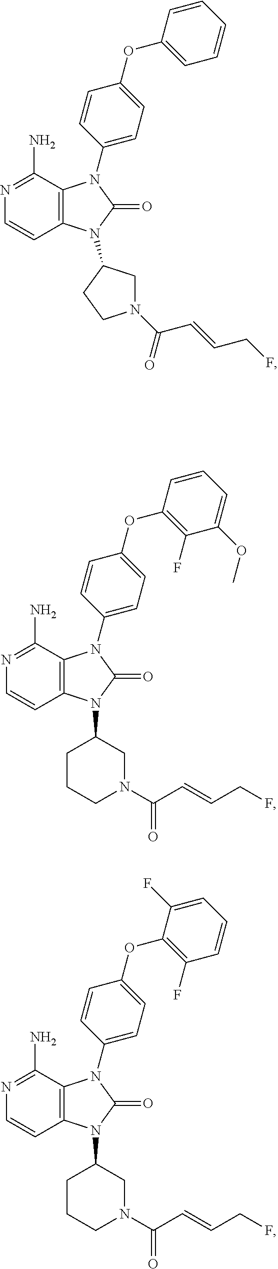 [SMILES: Nc1nccc2c1n(-c1ccc(Oc3ccccc3)cc1)c(=O)n2[C@H]1CCN(C(=O)/C=C/CF)C1 ; COc1cccc(Oc2ccc(-n3c(=O)n([C@@H]4CCCN(C(=O)/C=C/CF)C4)c4ccnc(N)c43)cc2)c1F ; Nc1nccc2c1n(-c1ccc(Oc3c(F)cccc3F)cc1)c(=O)n2[C@@H]1CCCN(C(=O)/C=C/CF)C1]

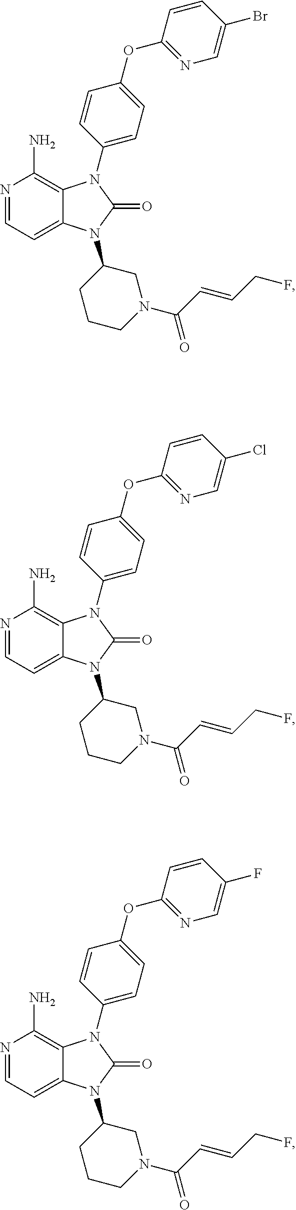 [SMILES: Nc1nccc2c1n(-c1ccc(Oc3ccc(Br)cn3)cc1)c(=O)n2[C@@H]1CCCN(C(=O)/C=C/CF)C1 ; Nc1nccc2c1n(-c1ccc(Oc3ccc(Cl)cn3)cc1)c(=O)n2[C@@H]1CCCN(C(=O)/C=C/CF)C1 ; Nc1nccc2c1n(-c1ccc(Oc3ccc(F)cn3)cc1)c(=O)n2[C@@H]1CCCN(C(=O)/C=C/CF)C1]

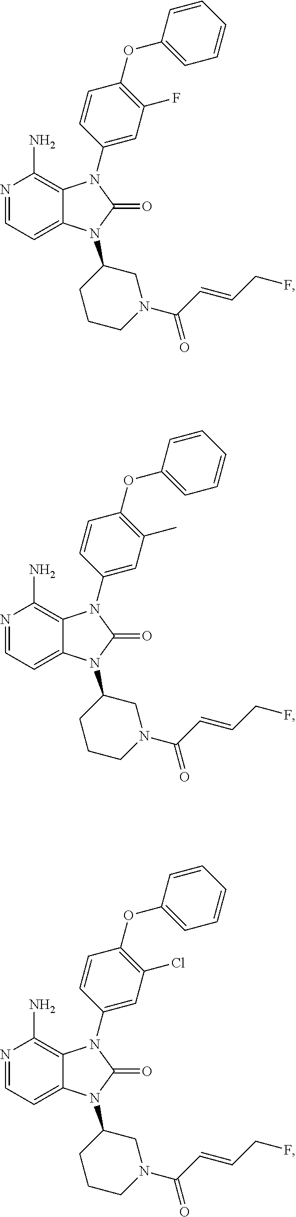 [SMILES: Nc1nccc2c1n(-c1ccc(Oc3ccccc3)c(F)c1)c(=O)n2[C@@H]1CCCN(C(=O)/C=C/CF)C1 ; Cc1cc(-n2c(=O)n([C@@H]3CCCN(C(=O)/C=C/CF)C3)c3ccnc(N)c32)ccc1Oc1ccccc1 ; Nc1nccc2c1n(-c1ccc(Oc3ccccc3)c(Cl)c1)c(=O)n2[C@@H]1CCCN(C(=O)/C=C/CF)C1]

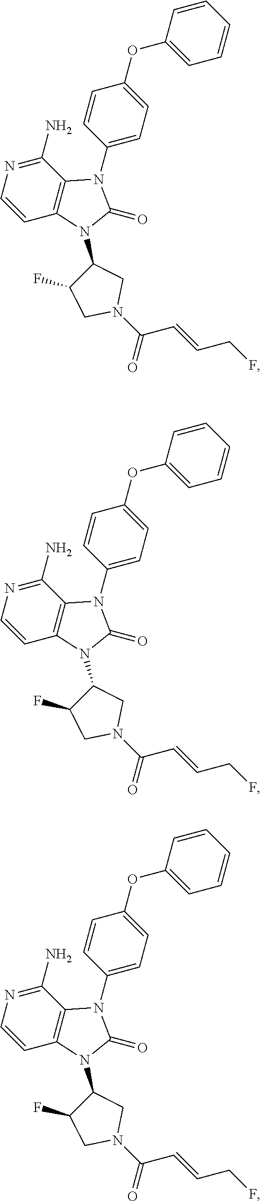 [SMILES: Nc1nccc2c1n(-c1ccc(Oc3ccccc3)cc1)c(=O)n2[C@H]1CN(C(=O)/C=C/CF)C[C@@H]1F ; Nc1nccc2c1n(-c1ccc(Oc3ccccc3)cc1)c(=O)n2[C@@H]1CN(C(=O)/C=C/CF)C[C@H]1F ; Nc1nccc2c1n(-c1ccc(Oc3ccccc3)cc1)c(=O)n2[C@H]1CN(C(=O)/C=C/CF)C[C@H]1F]

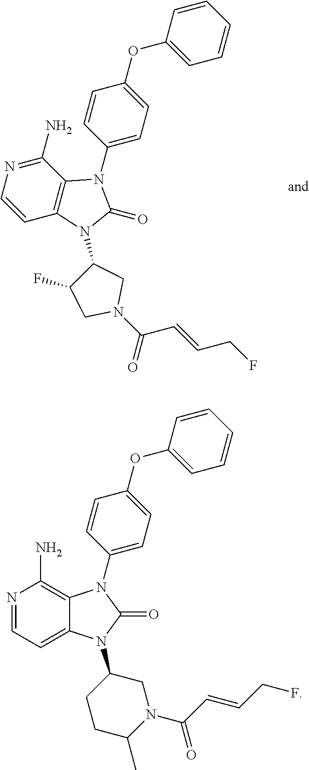 [SMILES: Nc1nccc2c1n(-c1ccc(Oc3ccccc3)cc1)c(=O)n2[C@@H]1CN(C(=O)/C=C/CF)C[C@@H]1F ; CC1CC[C@@H](n2c(=O)n(-c3ccc(Oc4ccccc4)cc3)c3c(N)nccc32)CN1C(=O)/C=C/CF]

### DEFINITION AND DESCRIPTION

Unless otherwise specified, the following terms and phrases used herein have the following meanings. A specific term or phrase should not be considered indefinite or unclear in the absence of a particular definition, but should be understood in the ordinary sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by contacting the compound with a sufficient amount of a base in a pure solution or a suitable inert solvent. The pharmaceutically acceptable base addition salt includes a salt of sodium, potassium, calcium, ammonium, organic amine, magnesium, or similar salts. When the compound of the present disclosure contains a relatively basic functional group, an acid addition salt can be obtained by contacting the compound with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid; and salts of amino acid (such as arginine), and a salt of an organic acid such as glucuronic acid. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by conventional chemical method. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereoisomers, (D)- isomers, (L)-isomers, racemic, and other mixtures thereof, such as enantiomers or diastereomer enriched mixtures, all of which are within the scope of the present disclosure. Additional asymmetric carbon atoms may be present in substituents such as alkyl. All these isomers and their mixtures are encompassed within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) or a straight dashed bond ( [symbol] ).

Unless otherwise specified, the terms “enriched in one isomer”, “enriched in isomers”, “enriched in one enantiomer”, or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomer, or D and L isomer can be prepared using chiral synthesis or chiral reagents or other conventional techniques. If one kind of enantiomer of certain compound of the present disclosure is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to give the pure enantiomer. In addition, the enantiomer and the diastereoisomer are generally isolated through chromatography which uses a chiral stationary phase and optionally combines with a chemical derivative method (such as carbamate generated from amine).

The compound of the present disclosure may contain an unnatural proportion of atomic isotope at one or more than one atom that constitute the compound. For example, the compound can be radiolabeled with a radioactive isotope, such as tritium (³H), iodine-125 (¹²⁵I), or C-14 (¹⁴C). For another example, deuterated drugs can be formed by replacing hydrogen with heavy hydrogen, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “optional” or “optionally” means that the subsequently described event or circumstance may, but does not necessarily, occur, and the description includes instances where the event or circumstance occurs and instances where it does not.

The term “substituted” means one or more than one hydrogen atom(s) on a specific atom are substituted by the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone. The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted with 0 to 2 R, the group can be optionally substituted with up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variables is selected from a single bond, it means that the two groups linked by the single bond are connected directly. For example, when L in A-L-Z represents a single bond, the structure of A-L-Z is actually A-Z.

When a substituent is vacant, it means that the substituent is absent, for example, when X is vacant in A-X, the structure of A-X is actually A. When the enumerative substituent does not indicate by which atom it is linked to the group to be substituted, such substituent can be bonded by any atom thereof. For example, when pyridyl acts as a substituent, it can be linked to the group to be substituted by any carbon atom on the pyridine ring. When the enumerative linking group does not indicate the direction for linking, the direction for linking is arbitrary, for example, the linking group L contained in

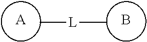

is -M-W-, then -M-W- can link ring A and ring B to form

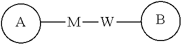

in the direction same as left-to-right reading order, and form

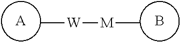

in the direction contrary to left-to-right reading order. A combination of the linking groups, substituents and/or variables thereof is allowed only when such combination can result in a stable compound.

Unless otherwise specified, the term “halide” or “halogen” by itself or as part of another substituent refer to fluorine (F), chlorine (Cl), bromine (Br) or iodine (I) atom.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁, and C₁₂, and any range from n to n+m is also included, for example C₁₋₁₂ includes C₁₋₃, C₁₋₆, C₁₋₉, C₃₋₆, C₃₋₉, C₃₋₁₂, C₆₋₉, C₆₋₁₂, and C₉₋₁₂; similarly, n-membered to n+m-membered means that the number of atoms on the ring is from n to n+m, for example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring, and any range from n to n+m is also included, for example, 3- to 12-membered ring includes 3- to 6-membered ring, 3- to 9-membered ring, 5- to 6-membered ring, 5- to 7-membered ring, 6- to 7-membered ring, 6- to 8-membered ring, and 6- to 10-membered ring.

Unless otherwise specified, the term “C₁₋₃ alkyl” refers to a linear or branched saturated hydrocarbon group consisting of 1 to 3 carbon atoms. The C₁₋₃ alkyl includes C₁₋₂, C₂₋₃ alkyl, etc.; it can be monovalent (such as methyl), divalent (such as methylene) or multivalent (such as methine). Examples of C₁₋₃ alkyl include, but are not limited to, methyl (Me), ethyl (Et), propyl (including n-propyl and isopropyl), etc.

Unless otherwise specified, the term “C₁₋₃ alkoxy” refers to an alkyl group containing 1 to 3 carbon atoms that are connected to the rest of the molecule through an oxygen atom. The C₁₋₃ alkoxy includes C₁₋₂, C₂₋₃, C₃, C₂ alkoxy, etc. Examples of C₁₋₃ alkoxy include, but are not limited to, methoxy, ethoxy, propoxy (including n-propoxy and isopropoxy), etc.

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the examples of the present disclosure.

The structure of the compounds of the present disclosure can be confirmed by conventional methods known to those skilled in the art, and if the present disclosure involves an absolute configuration of a compound, then the absolute configuration can be confirmed by means of conventional techniques in the art. For example, in the case of single crystal X-ray diffraction (SXRD), diffraction intensity data are collected from the cultured single crystal using a Bruker D8 venture diffractometer with CuKα radiation as the light source and scanning mode: φ/ω scan, and after collecting the relevant data, the crystal structure is further analyzed by direct method (Shelxs97), so that the absolute configuration can be confirmed.

The solvents used in the present disclosure are commercially available.

The compounds of the present disclosure are named according to the conventional naming principles in the art or by ChemDraw® software, and the commercially available compounds use the supplier catalog names.

### TECHNICAL EFFECT

The compound of the present invention has significant BTK enzyme inhibition activity and HPBMC cell activity; the compound of the present invention has excellent hepatocyte body stability and pharmacokinetic properties; and exhibits good medicinal efficacy in EAE mouse model.

### BRIEF DESCRIPTION OF THE DRAWINGS

FIG. 1. In vivo efficacy score of EAE in C57BL/6 mice.

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and specific embodiments thereof have also been disclosed; for those skilled in the art, it is obvious to make various modifications and improvements to the embodiments of the present disclosure without departing from the spirit and scope of the present disclosure.

### REFERENCE EXAMPLE 1. INTERMEDIATE A

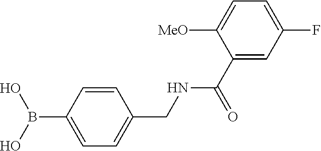 [SMILES: COc1ccc(F)cc1C(=O)NCc1ccc(B(O)O)cc1]

### Synthetic Route

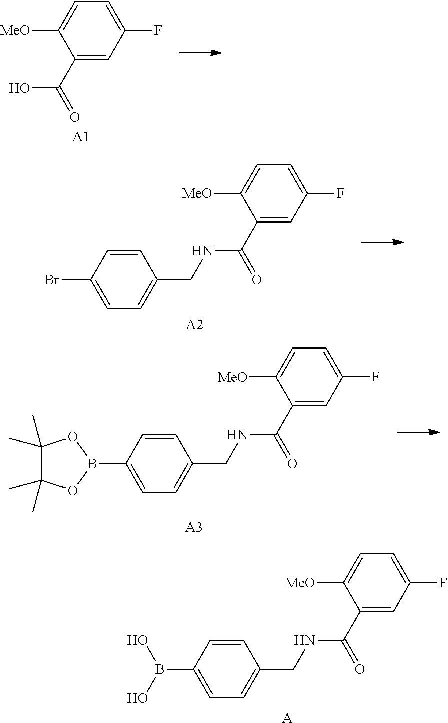

### Step 1: Synthesis of Compound A2

4-bromobenzoylamine (10 g, 55.75 mmol, 1.20 eq) was added to compound A1 (7.62 g, 44.79 mmol, 1.00 eq) and dichloromethane (100 mL), and 2-(7-azabenzotriazol-1-yl)-N,N,N′,N′-tetramethyluronium hexafluorophosphate (34 g, 89.6 mmol, 2 eq) and N,N-diisopropylethylamine (17.4 g, 134.4 mmol, 3 eq) were added. The mixture was reacted at 25° C. for 2 hours. The reaction solution was extracted with dichloromethane (70 mL*2), and the organic phases were combined, dried over anhydrous sodium sulfate, filtered and concentrated. The crude product was purified by column chromatography (petroleum ether: ethyl acetate) to obtain compound A2. LCMS: (ESI) m/z:337.9 [M+1]⁺.

### Step 2: Synthesis of Compound A3

The compound A2 (14 g, 41.4 mmol, 1.00 eq) was dissolved in 1,4-dichlorohexacyclo (200 mL) and water (40 mL), and bis(pinacolato)diboron (31.5 g, 124 mmol, 3.00 eq), potassium acetate (12.19 g, 124 mol, 3 eq) and [1,1′-bis(diphenylphosphino)ferrocene]dichloropalladium(II) (3.03 g, 4.14 mmol, 0.1 eq) were added in sequence. The reaction was heated and refluxed at 100° C. for 5 hours. The reaction solution was concentrated and water (100 mL) was added. The solution was extracted with ethyl acetate (50 mL*3), and the organic phases were combined, dried over anhydrous sodium sulfate, filtered and concentrated. The crude product was purified by column chromatography (petroleum ether: ethyl acetate) to obtain compound A3. LCMS: (ESI) m/z:386.0 [M+1]⁺.

### Step 3: Synthesis of Compound A

The compound A3 (2 g, 5.19 mmol, 1 eq) was dissolved in acetone (30 mL), and sodium periodate (3.33 g, 15.57 mmol, 863.04 μL, 3 eq) and ammonium acetate (1 M, 10.38 mL, 2 eq) were added. Nitrogen replacement for twice and the mixture was stirred at 20° C. for 19 hours. 5 mL of 4N HCl was added to the reaction solution system, and the mixture was stirred for 10 min, diluted with 30 mL of water, and extracted with ethyl acetate (50 mL*2). The organic phases were combined, dried over anhydrous sodium sulfate, filtered and concentrated. The crude product was purified by column chromatography (dichloromethane: methanol) to obtain compound A. LCMS: (ESI) m/z: 304.1 [M+1]⁺. ¹H NMR (400 MHz, DMSO-d₆) δ=8.78-8.75 (m, 1H), 7.85-7.71 (m, 2H), 7.52-7.48 (m, 1H), 7.36-7.28 (m, 3H), 7.19-7.16 (m, 1H), 4.53-4.49 (m, 2H), 3.88 (s, 3H).

### EXAMPLE 1

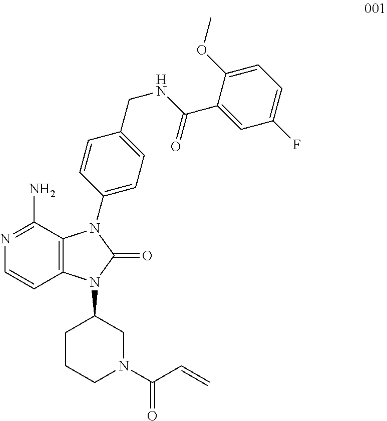 [SMILES: C=CC(=O)N1CCC[C@@H](n2c(=O)n(-c3ccc(CNC(=O)c4cc(F)ccc4OC)cc3)c3c(N)nccc32)C1]

### Synthetic Route

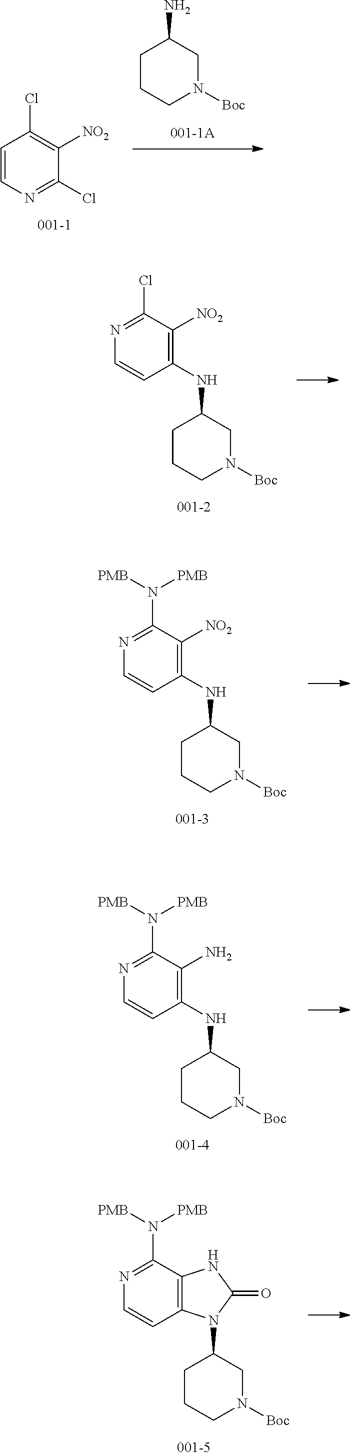

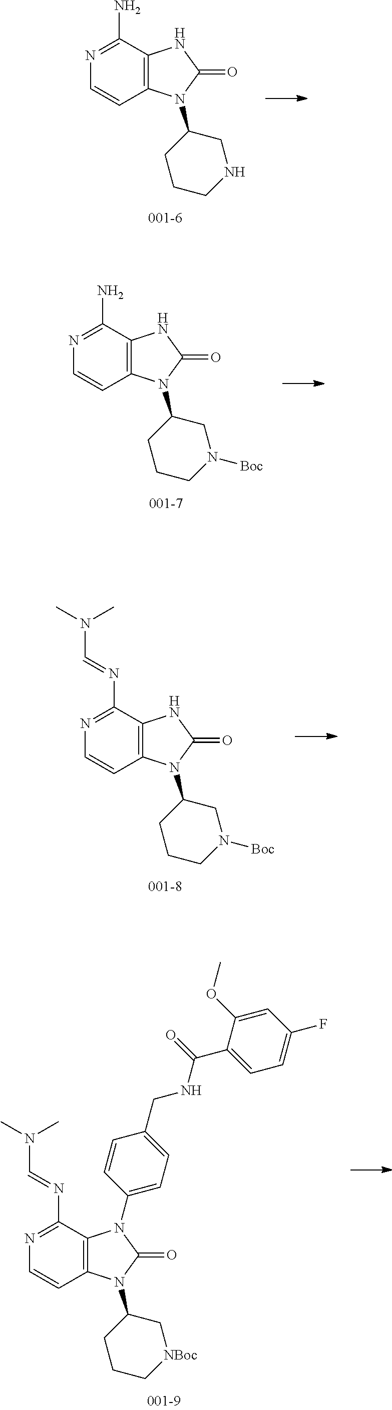 [SMILES: Nc1nccc2c1[nH]c(=O)n2[C@@H]1CCCNC1 ; CC(C)(C)OC(=O)N1CCC[C@@H](n2c(=O)[nH]c3c(N)nccc32)C1 ; CN(C)/C=N/c1nccc2c1[nH]c(=O)n2[C@@H]1CCCN(C(=O)OC(C)(C)C)C1 ; COc1cc(F)ccc1C(=O)NCc1ccc(-n2c(=O)n([C@@H]3CCCN(C(=O)OC(C)(C)C)C3)c3ccnc(/N=C/N(C)C)c32)cc1]

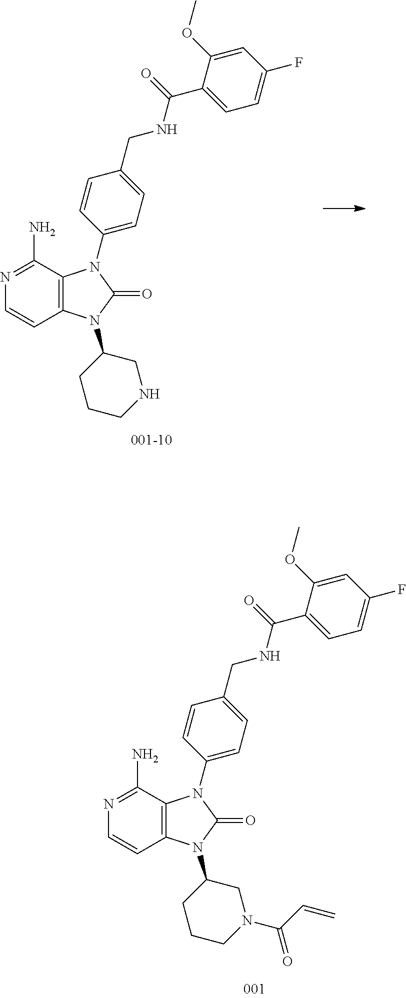 [SMILES: COc1cc(F)ccc1C(=O)NCc1ccc(-n2c(=O)n([C@@H]3CCCNC3)c3ccnc(N)c32)cc1 ; C=CC(=O)N1CCC[C@@H](n2c(=O)n(-c3ccc(CNC(=O)c4ccc(F)cc4OC)cc3)c3c(N)nccc32)C1]

### Step 1: Synthesis of Compound 001-2

The compound 001-1A (15 g, 74.90 mmol, 1 eq) and compound 001-1(14.45 g, 74.90 mmol, 1 eq) were dissolved in N,N-dimethylformamide (100 mL), triethylamine (11.37 g, 112.34 mmol, 15.64 mL, 1.5 eq) was added, and the mixture was reacted at 20° C. for 16 hours. After the reaction was completed, the reaction solution was diluted with 200 mL of water, and extracted with ethyl acetate (200 mL*3). The organic phases were combined, washed with saturated brine, and dried over anhydrous sodium sulfate to obtain a crude product. The crude product was purified by column chromatography (petroleum ether:ethyl acetate=to 1:1) to obtain compound 001-2. LCMS: (ESI) m/z:357.1 [M+1]⁺.

### Step 2: Synthesis of Compound 001-3

Bis(4-methoxybenzyl)amine (19.26 g, 74.83 mmol, 1 eq) and the compound 001-2(26.7 g, 74.83 mmol, 1 eq) were dissolved in isopropanol (300 mL), and triethylamine (9.84 g, 97.28 mmol, 13.54 mL, 1.3 eq) was added, and the mixture was reacted at 95° C. for 16 hours. The reaction solution was concentrated to obtain the compound 001-3. The compound 001-3 was used directly in the next step without purification. LCMS: (ESI) m/z: 578.4 [M+1]⁺.

### Step 3: Synthesis of Compound 001-4

The compound 001-3 (43 g, 74.44 mmol, 1 eq) was dissolved in acetic acid (200 mL) and methanol (200 mL), iron powder (49.88 g, 893.24 mmol, 12 eq) was added, and the mixture was reacted at 20° C. for 16 hours. The reaction solution was concentrated, adjusted to pH =8 with saturated sodium bicarbonate solution and extracted with dichloromethane (200 mL*3). The organic phases were washed with saturated sodium carbonate solution and dried over anhydrous sodium sulfate to obtain compound 001-4. The compound 001-4 was used directly in the next step without purification. LCMS: (ESI) m/z: 548.4 [M+1]⁺.

### Step 4: Synthesis of Compound 001-5

The compound 001-4 (23.7 g, 43.27 mmol, 1 eq) and 1-cyclohexyl-3-(2-morpholinoethyl)-carbodiimide metho-p-toluenesulfonate (10.52 g, 64.91 mmol, 1.5 eq) were dissolved in acetonitrile (230 mL), and the mixture was reacted at 80° C. for 5 hours. The reaction solution was concentrated to obtain a crude product. The crude product was purified by column chromatography (petroleum ether:ethyl acetate=to 1:5) to obtain compound 001-5. LCMS: (ESI) m/z: 574.4 [M+1]⁺.

### Step 5: Synthesis of Compound 001-6

The compound 001-5 (1.5 g, 2.61 mmol, 1 eq) was dissolved in dichloromethane (30 mL) and trifluoroacetic acid (30 mL), and reacted at 50° C. for 3 hours. The reaction solution was concentrated to obtain a trifluoroacetate salt of compound 001-6. The trifluoroacetate salt of compound 001-6 was used directly in the next step without purification. LCMS: (ESI) m/z: 233.9 [M+1]⁺.

### Step 6: Synthesis of Compound 001-7

a) The trifluoroacetate salt of compound 001-6 (600 mg, 2.57 mmol, 1 eq) and di-tert-butyl dicarbonate (617.51 mg, 2.83 mmol, 650.01 μL, 1.1 eq) were dissolved in 1,4-dioxane (15 mL), and sodium carbonate (954.17 mg, 9.00 mmol, 3.5 eq) was dissolved in water (15 mL) and added to the reaction solution. The mixture was reacted at 20° C. for 5 hours. Water was added, and the solution was extracted with dichloromethane (50 mL*2). The organic phases were combined and dried over anhydrous sodium sulfate. The solvent was removed by rotary evaporation and purified by a silica gel column (dichloromethane:methanol=50:1-30:1) to obtain compound 001-7. LCMS: (ESI) m/z: 333.9 [M+1]⁺.

### Step 7: Synthesis of Compound 001-8

The compound 001-7 (380 mg, 1.14 mmol, 1 eq) was dissolved in N,N-dimethylformamide dimethyl acetal (7 mL) and reacted at 40° C. for 8 hours. The reaction solution was filtered to obtain compound 001-8. LCMS: (ESI) m/z: 389.0 [M+1]⁺.

### Step 8: Synthesis of Compound 001-9

The compound 001-8 (270 mg, 695.05 μmol, 1 eq), copper acetate (63.12 mg, 347.52 μmol, 0.5 eq), triethylamine (281.33 mg, 2.78 mmol, 386.97 μL, 4 eq), 2,2,6,6-tetramethylpiperidinooxy (131.16 mg, 834.06 μmol, 1.2 eq) were dissolved in dichloromethane (20 mL), and oxygen (22.24 mg, 695.05 μmol, 1 eq) was introduced. The mixture was stirred for 0.25 hours and compound A (421.33 mg, 1.39 mmol, 2 eq) was added. The mixture was reacted at 25° C. for 25 hours. The reaction solution was filtered, and the filtrate was concentrated to obtain a crude product. The crude product was purified by column chromatography (dichloromethane:methanol=20:1-10:1) to obtain compound 001-9. LCMS: (ESI) m/z: 646.1 [M+1]⁺.

### Step 9: Synthesis of Compound 001-10

The compound 001-9 (490 mg, 758.84 μmol, 1 eq) was dissolved in 1,4-dioxane (18 mL), then hydrochloric acid solution (9.18 g, 65.46 mmol, 9 mL, 26% content, 86.27 eq) was added, and the mixture was reacted at 50° C. for 72 hours. pH was adjusted to 8 with sodium carbonate. The solution was extracted with dichloromethane (100 mL*3). The organic phases were combined, dried over anhydrous sodium sulfate and filtered, and the solvent was removed by rotary evaporation to obtain compound 001- 10. LCMS: (ESI) m/z: 491.0 [M+1]⁺.

### Step 10: Synthesis of Compound 001

The compound 001-10 (530 mg, 1.08 mmol, 1 eq) was dissolved in N,N-dimethylformamide (5 mL), and 2-(7-azabenzotriazol-1-yl)-N,N,N′,N′-tetramethyluronium hexafluorophosphate (616.24 mg, 1.62 mmol, 1.5 eq), N,N-diisopropylethylamine (418.92 mg, 3.24 mmol, 564.58 μL, 3 eq) and acrylic acid (62.29 mg, 864.37 μmol, 59.32 μL, 0.8 eq) were added. The mixture was reacted at 20° C. for 6 hours. The reaction solution was concentrated and the solvent was removed by rotary evaporation to obtain a crude product. The crude product was purified by machine separation (Chromatographic column: Phenomenex Gemini- NX 80*40 mm*3 μm; Mobile phase: [water (0.05% ammonia)-acetonitrile]; Acetonitrile %: 27%-57%, 8min) to obtain 001. LCMS: (ESI) m/z: 545.0 [M+1]⁺. ¹H NMR (400 MHz, CD₃Cl) δ ppm 1.68 (br s, 1 H) 1.80-2.21 (m, 4 H) 2.63 (br s, 1 H) 3.08-3.57 (m, 1 H) 3.82-4.35 (m, 7 H) 4.79 (br s, 3 H) 5.74 (br s, 1 H) 6.36 (br s, 1 H) 6.66 (br s, 2 H) 6.98 (br s, 1 H) 7.19 (s, 1 H) 7.41-7.70 (m, 3 H) 7.80-8.09 (m, 2 H) 8.37 (br s, 1 H).

### EXAMPLE 2

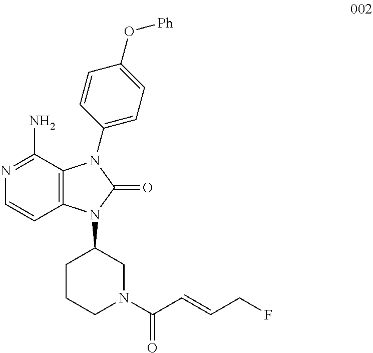 [SMILES: Nc1nccc2c1n(-c1ccc(Oc3ccccc3)cc1)c(=O)n2[C@@H]1CCCN(C(=O)/C=C/CF)C1]

### Synthetic Route

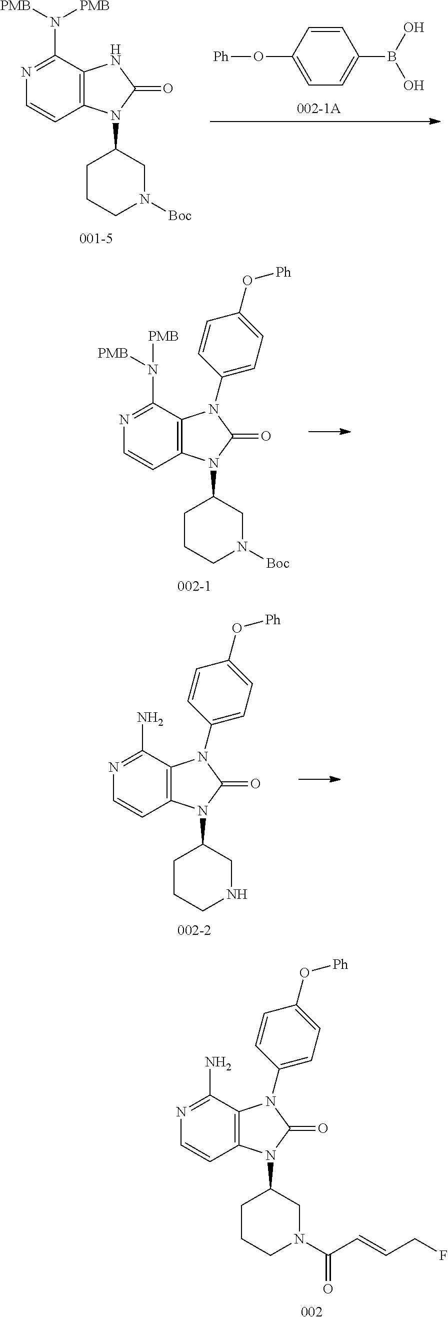

### Step 1: Synthesis of Compound 002-1

The compound 001-5 (7.46 g, 34.86 mmol, 2.5 eq) and 002-1A(7.46 g, 34.86 mmol, 2.5 eq) were dissolved in dichloromethane (80 mL), and triethylamine (5.64 g, 55.78 mmol, 7.76 mL, 4 eq) and 2,2,6,6-tetramethylpiperidinooxy (2.41 g, 15.34 mmol, 1.1 eq) were added, and finally copper acetate (1.27 g, 6.97 mmol, 0.5 eq) was added. The mixture was reacted at 25° C. for 48 hours under oxygen protection. The reaction solution was concentrated to obtain a crude product. The crude product was purified by column chromatography (petroleum ether:ethyl acetate=5:1) to obtain compound 002-1. LCMS: (ESI) m/z: 742.4 [M+1]⁺.

### Step 2: Synthesis of Compound 002-2

The compound 002-1 (0.17 g, 229.15 μmol, 1 eq) was dissolved in dichloromethane (3 mL), and trifluoroacetic acid (4.62 g, 40.52 mmol, 3 mL, 176.82 eq) was added, and the mixture was reacted at 50° C. for 4 hours. The reaction solution was concentrated to obtain a trifluoroacetate salt of compound 002-2. The trifluoroacetate salt of compound 002-2 was used directly in the next step without purification. LCMS: (ESI) m/z: 402.2 [M+1]⁺.

### Step 3: Synthesis of Compound 002

Propylphosphonic anhydride (72.92 mg, 114.58 μmol, 68.15 μL, 50%% content, 1 eq) was added to a mixture of the trifluoroacetate salt of compound 002-2 (0.046 g, 114.58 μmol, 1 eq), (E)-4-fluorobut-2-enoic acid (11.93 mg, 114.58 μmol, 1 eq), triethylamine (23.19 mg, 229.16 μmol, 31.90 μL, 2 eq) and N,N-dimethylformamide (2 mL) at 15° C., and the mixture was stirred for 1 hour after the addition. The reaction solution was concentrated to obtain a crude product. The crude product was separated by preparative HPLC (Chromatographic column: Phenomenex Gemini-NX 80*40 mm*3 μm; Mobile phase: [water (0.05% ammonia)-acetonitrile]; Acetonitrile %: 30%-60%, 8 min) to obtain compound 002. LCMS: (ESI) m/z: 488.2 [M+1]⁺. ¹H NMR (400 MHz, CD₃OD) δ ppm 1.31 (m, 1 H) 1.62-1.81 (m, 1 H) 1.95-2.16 (m, 2 H) 2.57 (br d, J=11.29 Hz, 1 H) 3.21-3.27 (m, 1 H) 3.52 (br s, 1 H) 4.29 (br s, 1 H) 4.60 (br s, 1 H) 5.15 (m, 2 H) 6.68-7.04 (m, 3 H) 7.08-7.27 (m, 5 H) 7.39-7.55 (m, 4 H) 7.79 (m, 1 H).

### EXAMPLE 3

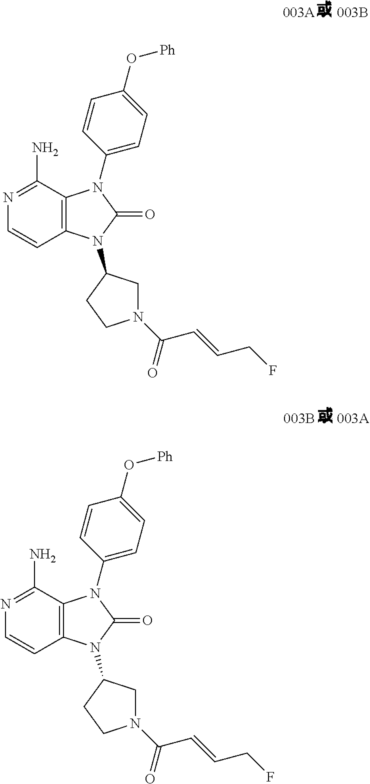 [SMILES: Nc1nccc2c1n(-c1ccc(Oc3ccccc3)cc1)c(=O)n2[C@@H]1CCN(C(=O)/C=C/CF)C1 ; Nc1nccc2c1n(-c1ccc(Oc3ccccc3)cc1)c(=O)n2[C@H]1CCN(C(=O)/C=C/CF)C1]

### Synthetic Route

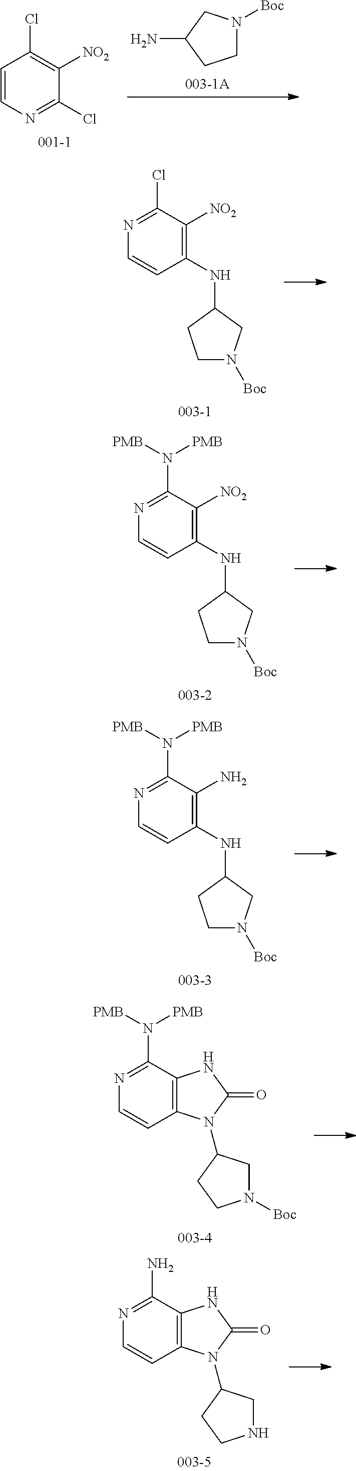

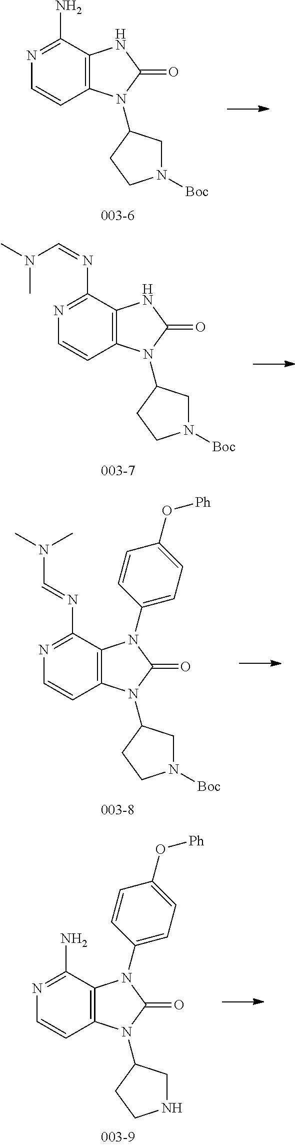 [SMILES: CC(C)(C)OC(=O)N1CCC(n2c(=O)[nH]c3c(N)nccc32)C1 ; CN(C)/C=N\c1nccc2c1[nH]c(=O)n2C1CCN(C(=O)OC(C)(C)C)C1 ; CN(C)/C=N/c1nccc2c1n(-c1ccc(Oc3ccccc3)cc1)c(=O)n2C1CCN(C(=O)OC(C)(C)C)C1 ; Nc1nccc2c1n(-c1ccc(Oc3ccccc3)cc1)c(=O)n2C1CCNC1]

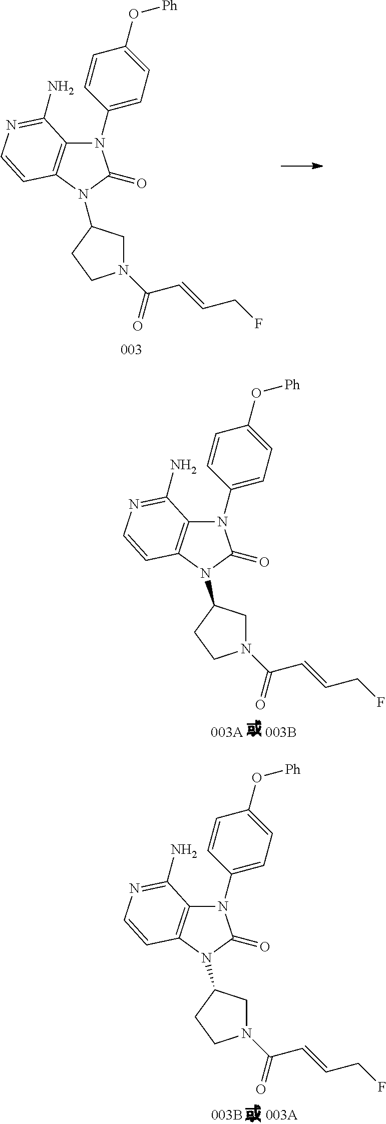 [SMILES: Nc1nccc2c1n(-c1ccc(Oc3ccccc3)cc1)c(=O)n2C1CCN(C(=O)/C=C/CF)C1 ; Nc1nccc2c1n(-c1ccc(Oc3ccccc3)cc1)c(=O)n2[C@@H]1CCN(C(=O)/C=C/CF)C1 ; Nc1nccc2c1n(-c1ccc(Oc3ccccc3)cc1)c(=O)n2[C@H]1CCN(C(=O)/C=C/CF)C1]

### Step 1: Synthesis of Compound 003-1

The compound 001-1 (10 g, 51.82 mmol, 1 eq) and 003-1A (9.65 g, 51.82 mmol, 4.39 mL, 1 eq) were dissolved in N,N-dimethylformamide (60 mL), then triethylamine (10.49 g, 103.63 mmol, 14.42 mL, 2 eq) was added, and the mixture was reacted at 25° C. for 10 hours. After the reaction was completed, water was added to the reaction solution. The solution was extracted with ethyl acetate and washed with saturated sodium chloride. The organic phases were combined and dried over anhydrous sodium sulfate to obtain a crude product 003-1. LCMS: (ESI) m/z: 342.9 [M+1]⁺.

### Step 2: Synthesis of Compound 003-2

Bis(4-methoxybenzyl)amine (12.01 g, 46.68 mmol, 1 eq) and the compound 003-1 (16.00 g, 46.68 mmol, 1 eq) were dissolved in isopropanol (150 mL), and triethylamine (6.14 g, 60.68 mmol, 8.45 mL, 1.3 eq) was added. The mixture was reacted at 95° C. for 5 hours. After the reaction was completed, the reaction solution was concentrated and dried by rotary evaporation, and purified by a silica gel column (petroleum ether:ethyl acetate=10:1-1:2) to obtain compound 003-2, and the compound 003-2 was directly used in the next step without purification. LCMS: (ESI) m/z: 564.3 [M+1]⁺.

### Step 3: Synthesis of Compound 003-3

The compound 003-2 (20.00 g, 35.48 mmol, 1 eq) was dissolved in methanol (100 mL), and acetic acid (100 mL) was added, then iron powder (19.72 g, 354.84 mmol, 10 eq) was added, and the mixture was reacted at 20° C. 16 hours. After the reaction was completed, it was carried out suction filtration using diatomaceous earth, and the reaction solution was concentrated, adjusted to pH=8 with saturated sodium bicarbonate solution, and extracted with dichloromethane (100 mL*3). The organic phases were washed with saturated sodium carbonate solution and dried over anhydrous sodium sulfate to obtain crude compound 003-3. LCMS: (ESI) m/z:534.3 [M+1]⁺.

### Step 4: Synthesis of Compound 003-4

The compound 003-3 (10.00 g, 18.74 mmol, 1 eq) and 1,1′-carbonyldiimidazole (9.12 g, 56.22 mmol, 3 eq) were dissolved in acetonitrile (100 mL), and the mixture was refluxed at 90° C. for 8 hours. After the reaction was completed, the solvent was removed by vacuum distillation and purified by silica gel column chromatography (petroleum ether:ethyl acetate=5:1-1:2) to obtain compound 003-4, LCMS: (ESI) m/z: 560.1 [M+1]⁺.

### Step 5: Synthesis of Compound 003-5

The compound 003-4 (4.8 g, 8.58 mmol, 1 eq) was dissolved in dichloromethane (5 mL), then trifluoroacetic acid (28.16 g, 246.97 mmol, 18.29 mL, 28.80 eq) was added, and the mixture was reacted at 50° C. for 8 hours after the addition. After the reaction was completed, the solvent was directly removed by rotary evaporation to obtain a trifluoroacetate salt of crude compound 003-5, which was used directly in the next step without purification. LCMS: (ESI) m/z: 219.9 [M+1]⁺.

### Step 6: Synthesis of Compound 003-6

The trifluoroacetate salt of compound 003-5 (1.8 g, 8.21 mmol, 1 eq) was dissolved in water (20 mL) and sodium carbonate (2.61 g, 24.63 mmol, 3 eq) was added. The mixture was stirred, and 1,4-dioxane (20 mL) was added, and finally di-tert-butyl dicarbonate (1.79 g, 8.21 mmol, 1.89 mL, 1 eq) was added. The mixture was stirred at 20° C. for 2 hours, and the solvent was removed under reduced pressure after the reaction was completed. The crude product was purified by silica gel column chromatography (dichloromethane:methanol=0%−10%) to obtain compound 003-6, LCMS: (ESI) m/z: 319.9 [M+1]⁺.

### Step 7: Synthesis of Compound 003-7

The compound 003-6(2.30 g, 7.20 mmol, 1 eq) was dissolved in N,N- dimethylformamide dimethyl acetal(15 mL), then the mixture was heated to 50° C. and reacted for 2 hours. After the reaction was completed, it was cooled to room temperature and solid precipitate was generated. The mixture was carried out suction filtration to obtain compound 003-7. LCMS: (ESI) m/z: 375.0 [M+1]⁺.

### Step 8: Synthesis of Compound 003-8

The compound 003-7 (1 g, 2.67 mmol, 1 eq), 002-1A (857.39 mg, 4.01 mmol, 1.5 eq), copper acetate (242.54 mg, 1.34 mmol, 0.5 eq) and 2,2,6,6 -tetramethylpiperidinooxy (503.97 mg, 3.20 mmol, 1.2 eq) were dissolved in dichloromethane (10 mL), then triethylamine (1.35 g, 13.35 mmol, 1.86 mL, 5 eq) was added, and the mixture was stirred at 20° C. for 16 hours. After the reaction was completed, the solvent was removed under reduced pressure and purified by a silica gel column (petroleum ether:ethyl acetate=10:1-1:2) to obtain compound 003-8. LCMS: (ESI) m/z: 543.1 [M+H]⁺.

### Step 9: Synthesis of Compound 003-9

The compound 003-8 (0.5 g, 921.44 μmol, 1 eq) was dissolved in 1,4-dioxane (3 mL), then hydrochloric acid (12 M, 3.84 mL, 50 eq) was added, and the mixture was reacted at 75° C. for 48 hours after the addition. After the reaction was completed, the solvent was removed under reduced pressure to obtain a hydrochloride of crude compound 003-9. LCMS:(ESI) m/z: 387.9 [M+1]⁺.

### Step 10: Synthesis of Compound 003

The hydrochloride of compound 003-9 (600.00 mg, 1.55 mmol, 1 eq) and (E)-4-fluorobut-2-enoic acid (161.18 mg, 1.55 mmol, 1 eq) were dissolved in N,N-dimethylformamide (10 mL), then triethylamine (313.41 mg, 3.10 mmol, 431.10 μL, 2 eq) was added, and finally propylphosphonic anhydride (1.48 g, 4.65 mmol, 1.38 mL, 3 eq) was added, and the mixture was stirred at 20° C. for 2 hours after addition. After the reaction was completed, the solvent was removed under reduced pressure. The crude product was purified by column chromatography (dichloromethane:methanol=100:1-10:1) to obtain compound 003. The compound 003 was separated by SFC, chromatographic column: (s,s) WHELK-O1 (250 mm*30 mm, 5 μm); mobile phase: [0.1% ammonia-methanol]; methanol %: 50%-50%, min, 2 compounds, 003A and 003B, were separated.

The analysis and characterization of compound 003A were as follows:

### SFC Analysis Method:

Column: (S,S)Whelk-01 100×4.6mm I.D., 5.0 μm; Mobile phase: A (CO₂) and B (methanol, containing 0.05% diethylamine); Gradient: B %=60%; Flow rate: 2.5 mL/min; Wavelength: 220 nm; Pressure: 100 bar, Rt=3.96 min.

LCMS: (ESI) m/z: 473.9 [M+H]⁺, ¹H NMR (400 MHz, CDCl₃) δ ppm 2.21-2.41 (m, 1 H) 2.52-2.74 (m, 1 H) 3.49-3.72 (m, 1 H) 3.92-4.00 (m, 2 H) 4.08 (br d, J=5.27 Hz, 2 H) 4.94-5.11 (m, 2 H) 6.27-6.44 (m, 1 H) 6.49 (br dd, J=11.04, 5.52 Hz, 1 H) 6.84-6.99 (m, 1 H) 7.04 (br t, J=8.53 Hz, 4 H) 7.12 (br t, J=7.28 Hz, 1 H) 7.27-7.40 (m, 4 H) 7.79 (br dd, J=9.03, 5.52 Hz, 1 H).

The analysis and characterization of compound 003B are as follows:

### SFC Analysis Method:

Column: (S,S)Whelk-01 100×4.6mm I.D., 5.0 um; Mobile phase: A (CO₂) and B (methanol, containing 0.05% diethylamine); Gradient: B%=60%; Flow rate: 2.5 mL/min; Wavelength: 220 nm; Pressure: 100 bar, Rt=4.88 min.

LCMS: (ESI) m/z: 473.9 [M+H]⁺, ¹H NMR (400 MHz, CDCl₃) δ ppm 2.21-2.41 (m, 1 H) 2.52-2.74 (m, 1 H) 3.49-3.72 (m, 1 H) 3.92-4.00 (m, 2 H) 4.08 (br d, J=5.27 Hz, 2 H) 4.94-5.11 (m, 2 H) 6.27-6.44 (m, 1 H) 6.49 (br dd, J=11.04, 5.52 Hz, 1 H) 6.84-6.99 (m, 1 H) 7.04 (br t, J=8.53 Hz, 4 H) 7.12 (br t, J=7.28 Hz, 1 H) 7.27-7.40 (m, 4 H) 7.79 (br dd, J=9.03, 5.52 Hz, 1 H).

### EXAMPLE 4

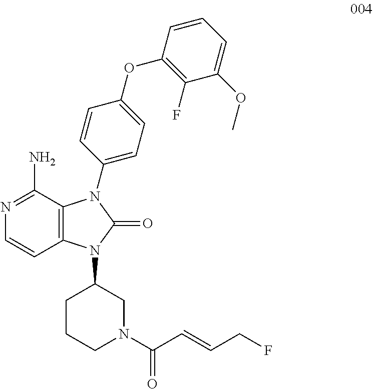 [SMILES: COc1cccc(Oc2ccc(-n3c(=O)n([C@@H]4CCCN(C(=O)/C=C/CF)C4)c4ccnc(N)c43)cc2)c1F]

### Synthetic Route

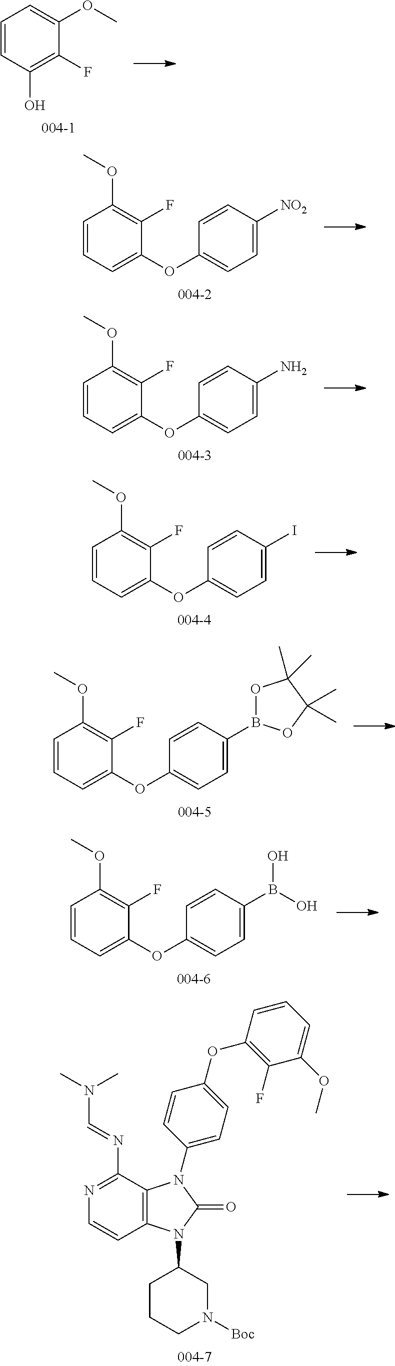

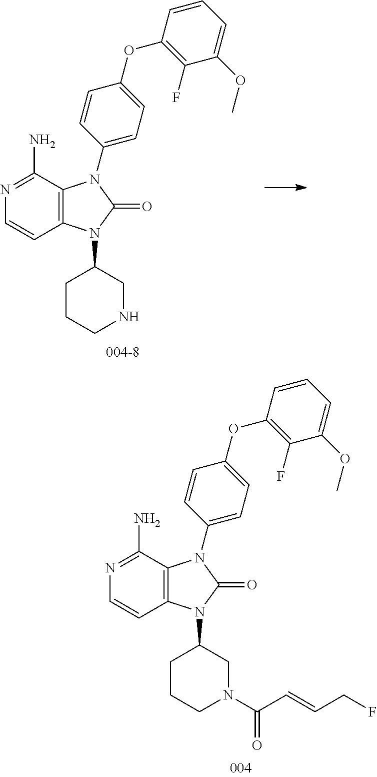 [SMILES: COc1cccc(Oc2ccc(-n3c(=O)n([C@@H]4CCCNC4)c4ccnc(N)c43)cc2)c1F ; COc1cccc(Oc2ccc(-n3c(=O)n([C@@H]4CCCN(C(=O)/C=C/CF)C4)c4ccnc(N)c43)cc2)c1F]

### Step 1: Synthesis of Compound 004-2

The compound 004-1 (1 g, 7.04 mmol, 1 eq) and p-fluoronitrobenzene (1.19 g, 8.44 mmol, 895.73 μL, 1.2 eq) were dissolved in acetonitrile (30 mL), and potassium carbonate (2.92 g, 21.11 mmol, 3 eq) was added. The mixture was reacted at 90° C. for 16 hours, and carried out suction filtration after the reaction was completed. The solvent of the filtrate was removed by rotary evaporation, and the crude product was purified by a silica gel column (petroleum ether:ethyl acetate=10:1-1:1) to obtain compound 004-2. ¹H NMR (400 MHz, CDCl₃) δ ppm 3.9-4.0 (m, 3 H) 6.7-6.8 (m, 1 H) 6.9-7.0 (m, 1 H) 7.0-7.1 (m, 2 H) 7.1-7.2 (m, 1 H) 8.2-8.3 (m, 2 H).

### Step 2: Synthesis of Compound 004-3

The compound 004-2 (1.8 g, 6.84 mmol, 1 eq) was dissolved in ethanol (10 mL), then hydrochloric acid (2 M, 2 mL, 5.85e-1 eq) and iron powder (1.91 g, 34.19 mmol, 5 eq) were added. The mixture was reacted at 90° C. for 2 hours, and carried out suction filtration after the reaction was completed. The solvent of the filtrate was removed by rotary evaporation to obtain crude compound 004-3, which was used directly in the next step. ¹H NMR (400 MHz, CDCl₃) δ ppm 3.8 (s, 3 H) 6.4-6.5 (m, 1 H) 6.5-6.7 (m, 3 H) 6.8 (br d, J=8.8 Hz, 2 H) 6.8-6.9 (m, 1 H).

### Step 3: Synthesis of Compound 004-4

The 004-3 (1.09 g, 4.67 mmol, 1 eq) was dissolved in hydrochloric acid (2.30 g, 23.37 mmol, 2.26 mL, 37% content, 5 eq). The mixture was stirred at 0-5° C. for 30 minutes, 5 mL of sodium nitrite solution (1M) was added to the reaction solution, and the mixture continued to be stirred for 30 minutes, then potassium iodide (3.88 g, 23.37 mmol, 5 eq) was added, and the mixture continued to be stirred at 25° C. for 30 minutes. After the reaction was completed, it was extracted with ethyl acetate 3 times, 50 mL each time, and the organic phases were combined, washed with 10% sodium sulfite solution (20 mL), dried over anhydrous sodium sulfate, and the solvent removed by rotary evaporation, and the crude product was purified by column chromatography (petroleum ether) to obtain compound 004-4. ¹H NMR (400 MHz, CDCl₃) δ ppm 3.9 (s, 3 H) 6.7 (ddd, J=8.3, 7.0, 1.5 Hz, 1 H) 6.8-6.8 (m, 2 H) 6.8-6.8 (m, 1 H) 7.0-7.1 (m, 1 H) 7.6-7.6 (m, 2 H).

### Step 4: Synthesis of Compound 004-5

004-4 (0.9 g, 2.62 mmol, 1 eq) and bis(pinacolato)diboron (996.21 mg, 3.92 mmol, 1.5 eq) were dissolved in 1,4-dioxane (15 mL), then potassium carbonate (1.08 g, 7.85 mmol, 3 eq) and [1,1′- bis(diphenylphosphino)ferrocene]dichloropalladium(II) (382.74 mg, 523.07 μmol, 0.2 eq) were added, and the mixture was reacted at 100° C. for 5 hours under nitrogen protection. The solvent was directly removed by rotary evaporation after the reaction was completed, and the crude product was purified by column chromatography (petroleum ether 100%) to obtain 004-5. ¹H NMR (400 MHz, CDCl₃) δ ppm 1.23-1.28 (d, J=3.5 Hz, 12 H) 3.8 (d, J=3.8 Hz, 3 H) 6.5-6.6 (m, 1 H) 6.6-6.8 (m, 1 H) 6.8-7.0 (m, 3 H) 7.6-7.8 (m, 2 H).

### Step 5: Synthesis of Compound 004-6

The 004-5 (0.33 g, 958.79 μmol, 1 eq) was dissolved in acetone (8 mL) and water (2 mL), sodium periodate (615.23 mg, 2.88 mmol, 159.39 μL, 3 eq) and ammonium acetate (147.81 mg, 1.92 mmol, 2 eq) were added. After the reaction was completed, it was carried out suction filtration to remove solid impurities, and the filtrate was concentrated under reduced pressure to obtain crude compound 004-6. ¹H NMR (400 MHz, CDCl₃) δ ppm 4.0-4.4 (m, 3 H) 6.4-7.1 (m, 5 H) 7.6-8.1 (m, 2 H).

### Step 6: Synthesis of Compound 004-7

a) Copper acetate (116.89 mg, 643.56 μmol, 0.5 eq) and 2,2,6,6-tetramethylpiperidinooxy (242.88 mg, 1.54 mmol, 1.2 eq) were dissolved in dichloromethane (4 mL), and triethylamine (520.98 mg, 5.15 mmol, 716.61 μL, 4 eq), 001-8 (0.5 g, 1.29 mmol, 1 eq) and 004-6 (505.92 mg, 1.93 mmol, 1.5 eq) were added, under oxygen protection, the mixture was stirred at 25° C. for 8 hours. After the reaction was completed, the solvent of the reaction solution was removed by rotary evaporation, and the crude product was purified by column chromatography (ethyl acetate:petroleum ether=10%-100%) to obtain compound 004-7. LCMS: (ESI) m/z: 605.1 [M+1]⁺.

### Step 7: Synthesis of Compound 004-8

The 004-7 (0.5 g, 826.90 μmol, 1 eq) was dissolved in 1,4-dioxane (2 mL), and hydrochloric acid (12 M, 68.91 μL, 1 eq) was added, and the mixture was stirred at 70° C. for 56 hours. After the reaction was completed, the reaction solution was cooled to room temperature and the solvent was removed by rotary evaporation to obtain a hydrochloride of compound 004-8. LCMS: (ESI) m/z: 449.9 [M+1]⁺.

### Step 8: Synthesis of Compound 004

The hydrochloride salt of compound 004-8 (100 mg, 222.48 μmol, 1 eq) and (E)-4-fluorobut-2-enoic acid (23.16 mg, 222.48 μmol, 1 eq) were dissolved in N,N-dimethylformamide (2 mL), and triethylamine (67.54 mg, 667.44 μmol, 92.90 μL, 3 eq) and propylphosphonic anhydride (283.16 mg, 444.96 μmol, 264.63 μL, 2 eq) were added. The mixture was stirred at 25° C. for 4 hours. After the reaction was completed, the reaction solution was concentrated to obtain a crude product. Preparative separation of the crude product: chromatographic column: Phenomenex C18 80*40 mm*3 μm; mobile phase: [water (ammonia)-acetonitrile]; acetonitrile %: 38%-68%, 8 min, to obtain compound 004. LCMS: (ESI) m/z: 536.1 [M+1]⁺.

### EXAMPLE 5

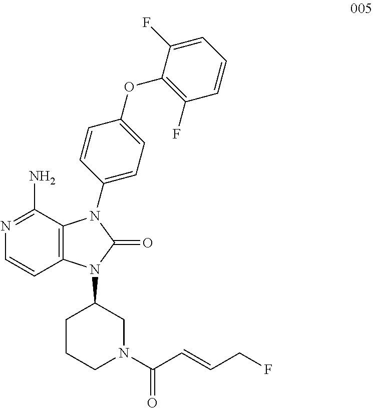 [SMILES: Nc1nccc2c1n(-c1ccc(Oc3c(F)cccc3F)cc1)c(=O)n2[C@@H]1CCCN(C(=O)/C=C/CF)C1]

### Synthetic Route

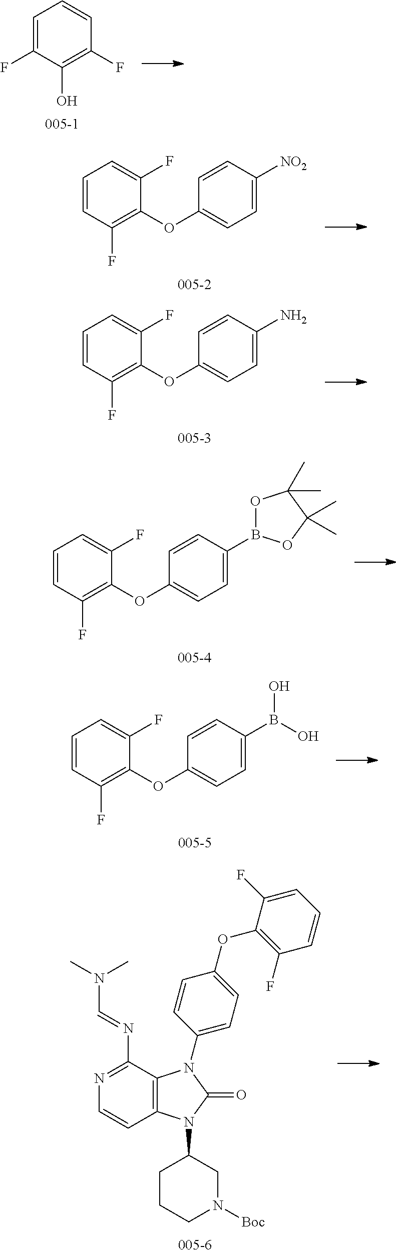

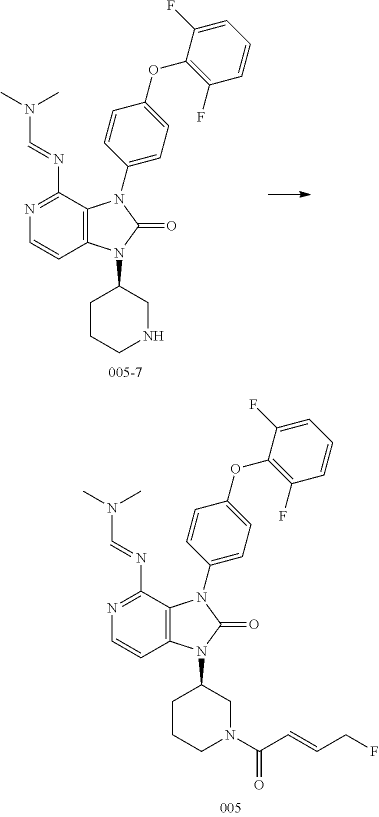 [SMILES: CN(C)/C=N/c1nccc2c1n(-c1ccc(Oc3c(F)cccc3F)cc1)c(=O)n2[C@@H]1CCCNC1 ; CN(C)/C=N/c1nccc2c1n(-c1ccc(Oc3c(F)cccc3F)cc1)c(=O)n2[C@@H]1CCCN(C(=O)/C=C/CF)C1]

### Step 1: Synthesis of Compound 005-2

The compound 005-1 (3.13 g, 24.10 mmol, 2 eq), p-fluoronitrobenzene (1.7 g, 12.05 mmol, 1.28 mL, 1 eq) were dissolved in acetonitrile (30 mL), and potassium carbonate (5.00 g, 36.14 mmol, 3 eq) was added, and the mixture was reacted at 90° C. for 16 hours and filtered after the reaction was completed. The solvent was removed by rotary evaporation, and the crude product was purified by a silica gel column (developing solvent: petroleum ether) to obtain compound 005-2. ¹H NMR (400 MHz, CDCl₃) 8 ppm 6.9-7.0 (m, 4 H) 7.1-7.2 (m, 1 H) 8.1-8.2 (m, 2 H).

### Step 2: Synthesis of Compound 005-3

The compound 005-2 (10.36 g, 41.24 mmol, 1 eq) was dissolved in ethanol (60 mL), then hydrochloric acid (2 M, 2 mL, 3.88e-1 eq) and iron powder (11.52 g, 206.22 mmol, 5 eq) were added, and the mixture was reacted at 90° C. for 2 hours and filtered after the reaction was completed. The solvent of the filtrate was directly removed by rotary evaporation to obtain crude compound 005-3. LCMS:(ESI) m/z:221.8[M+1]⁺; ¹H NMR (400 MHz, CDCl₃) δ ppm 2.9-4.0 (m, 2 H) 6.6-6.7 (m, 2 H) 6.8-6.9 (m, 2 H) 6.9-7.0 (m, 2 H) 7.1-7.2 (m, 1 H).

### Step 3: Synthesis of Compound 005-4

The compound 005-3 (1 g, 4.52 mmol, 1 eq), dibenzoyl peroxide (54.75 mg, 226.04 μmol, 0.05 eq) and bis(pinacolato)diboron (1.38 g, 5.42 mmol, 1.2 eq) were dissolved in acetonitrile (10 mL), and the mixture was stirred at 0-5° C. for 5 minutes. Tert-butyl nitrite (699.27 mg, 6.78 mmol, 806.54 μL, 1.5 eq) was added at 0° C., and the mixture continued to be stirred at 25° C. for 7 hours. After the reaction was completed, the solvent was removed by rotary evaporation, and the crude product was purified by a silica gel column (developing solvent: petroleum ether) to obtain compound 005-4. ¹H NMR (400 MHz, CDCl₃) δ ppm 1.3 (s, 12 H) 6.8 (d, J=8.5 Hz, 2 H) 6.9 - 7.0 (m, 2 H) 7.1-7.1 (m, 1 H) 7.7 (d, J=8.5 Hz, 2 H).

### Step 4: Synthesis of Compound 005-5

The compound 005-4 (4 g, 12.04 mmol, 1 eq) was dissolved in acetone (80 mL) and water (20 mL), and sodium periodate (7.73 g, 36.13 mmol, 2.00 mL, 3 eq) and ammonium acetate (1.86 g, 24.09 mmol, 2 eq) were added. The mixture was reacted at 25° C. for 24 hours, and the acetone was removed by rotary evaporation after the reaction was completed. The pH of the aqueous phase was adjusted to 5-6 with dilute hydrochloric acid. The mixture was extracted with ethyl acetate 3 times, 50 mL each time, and washed with saturated sodium chloride. The organic phases were combined and dried over anhydrous sodium sulfate, and the solvent was removed by rotary evaporation. The crude product was purified by column chromatography (petroleum ether:ethyl acetate=5:1-1:2) to obtain compound 005-5. ¹H NMR (400 MHz, CDCl₃) 8 ppm 6.9-7.0 (m, 4 H) 7.1-7.2 (m, 1 H) 8.1 (d, J=8.5 Hz, 2 H).

### Step 5: Synthesis of Compound 005-6

Copper acetate (58.45 mg, 321.78 μmol, 0.5 eq) and 2,2,6,6-tetramethylpiperidinooxy (121.44 mg, 772.27 μmol, 1.2 eq) were dissolved in dichloromethane (4 mL), then triethylamine (260.49 mg, 2.57 mmol, 358.31 μL, 4 eq), compound 001-8 (0.25 g, 643.56 μmol, 1 eq) and compound 005-5 (241.34 mg, 965.34 μmol, 1.5 eq) were added, under oxygen protection, the mixture was stirred at 25° C. for 8 hours. After the reaction was completed, the solvent was directly removed by rotary evaporation and the crude product was purified by column chromatography (petroleum ether:ethyl acetate=10%-100%) to obtain compound 005-6. LCMS: (ESI) m/z: 593.1 [M+1]⁺.

### Step 6: Synthesis of Compound 005-7

The compound 005-6 (0.4 g, 674.95 μmol, 1 eq) was dissolved in 1,4-dioxane (2 mL), and hydrochloric acid (12 M, 2 mL, 35.56 eq) was added. The mixture was stirred at 70° C. for 56 hours. After the reaction was completed, the solvent was directly removed by rotary evaporation to obtain a hydrochloride of the compound 005-7. LCMS: (ESI) m/z: 438.0 [M+1]⁺.

### Step 7: Synthesis of Compound 005

The hydrochloride salt of compound 005-7 (100 mg, 228.60 μmol, 1 eq) and (E)-4-fluorobut-2-enoic acid (23.79 mg, 228.60 μmol, 1 eq) were dissolved in N,N-dimethylformamide (2 mL), and triethylamine (69.40 mg, 685.81 μmol, 95.46 μL, 3 eq) and propylphosphonic anhydride (290.95 mg, 457.20 μmol, 271.91 μL, 50% content, 2 eq) were added. The mixture was stirred at 25° C. for 4 hours. After the reaction was completed, the reaction solution was concentrated, and the crude product was carried out preparative separation: chromatographic column: Phenomenex C18 80*40 mm*3 μm; mobile phase: [water (ammonia)-acetonitrile]; acetonitrile%: 38%-68%, 8 min, to obtain compound 005. LCMS:(ESI) m/z:524.1 [M+1]⁺; ¹H NMR (400 MHz, CDCl₃) δ ppm 1.82-2.06 (m, 3 H) 2.33-2.70 (m, 1 H) 2.92-3.19 (m, 0.5 H) 3.49 (s, 0.5 H) 3.74-3.93 (m, 1 H) 4.02-4.21 (m, 2 H) 4.61-4.81 (m, 1 H) 4.90 - 4.99 (m, 1 H) 5.03-5.13 (m, 1 H) 6.44-6.60 (m, 2 H) 6.78-6.92 (m, 1 H) 6.95-7.00 (m, 2 H) 7.02 (br d, J=8.5 Hz, 2 H) 7.10-7.17 (m, 1 H) 7.32 (br d, J=8.8 Hz, 2 H) 7.72-7.89 (m, 1 H).

### EXAMPLE 6

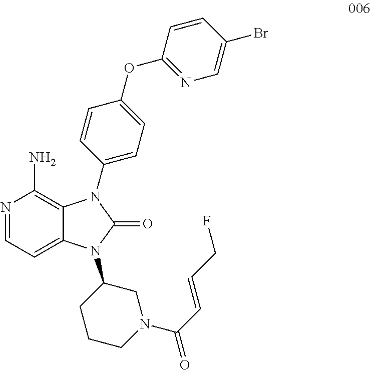 [SMILES: Nc1nccc2c1n(-c1ccc(Oc3ccc(Br)cn3)cc1)c(=O)n2[C@@H]1CCCN(C(=O)/C=C/CF)C1]

### Synthetic Route

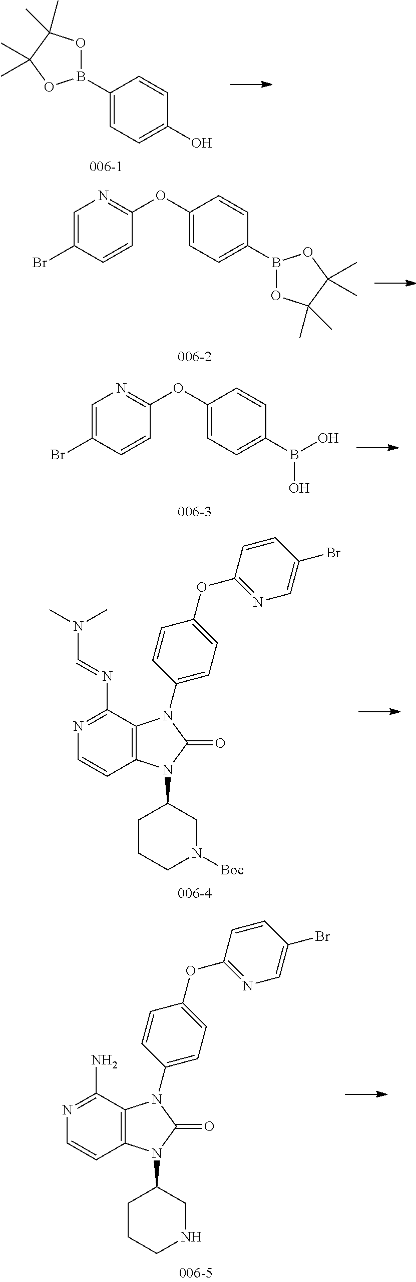

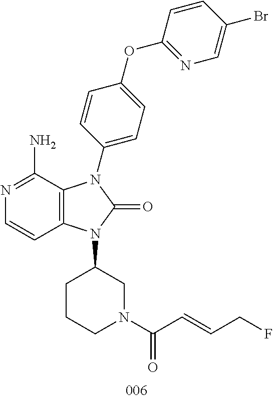 [SMILES: Nc1nccc2c1n(-c1ccc(Oc3ccc(Br)cn3)cc1)c(=O)n2[C@@H]1CCCN(C(=O)/C=C/CF)C1]

### Step 1: Synthesis of Compound 006-2

The compound 006-1 (2 g, 9.09 mmol, 1 eq), 2-fluoro-5-bromopyridine (1.60 g, 9.09 mmol, 935.29 μL, 1 eq) were dissolved in acetonitrile (20 mL), and potassium carbonate (2.51 g, 18.18 mmol, 2 eq) was added, and the mixture was reacted at 90° C. for 10 hours. After the reaction was completed, it was carried out suction filtration, and water was added to the filtrate. The mixture was extracted with ethyl acetate twice, 50 mL each time, washed with saturated sodium chloride. The organic phases were combined and dried over anhydrous sodium sulfate. The solvent was removed by rotary evaporation, and the crude product was purified by a silica gel column (developing solvent: petroleum ether) to obtain compound 006-2. LCMS: (ESI) m/z: 375.8 [M+1]⁺.

### Step 2: Synthesis of Compound 006-3

The compound 006-2 (2.4 g, 6.38 mmol, 1 eq) was dissolved in acetone (20 mL) and water (5 mL), and sodium periodate (4.10 g, 19.15 mmol, 1.06 mL, 3 eq) and ammonium acetate (983.90 mg, 12.76 mmol, 2 eq) were added, and the mixture was reacted at 25° C. for 24 hours. After the reaction was completed, the acetone was removed by rotary evaporation, and the pH of the aqueous phase was adjusted to 5-6 with dilute hydrochloric acid. The mixture was extracted with ethyl acetate 3 times, 50 mL each time, and washed with saturated sodium chloride. The organic phases were combined and dried over anhydrous sodium sulfate. The solvent was removed by rotary evaporation and the crude product was purified by column chromatography (petroleum ether:ethyl acetate =5:1-1:2) to obtain compound 006-3. LCMS: (ESI) m/z: 293.8 [M+1]⁺.

### Step 3: Synthesis of Compound 006-4

Copper acetate (11.69 mg, 64.36 μmol, 0.5 eq) and 2,2,6,6-tetramethylpiperidinooxy (24.29 mg, 154.45 μmol, 1.2 eq) were dissolved in dichloromethane (2 mL), and triethylamine (52.10 mg, 514.85 μmol, 71.66 μL, 4 eq), 006-3 (0.05 g, 128.71 μmol, 1 eq) and 001-8 (56.74 mg, 193.07 μmol, 1.5 eq) were added. The mixture was stirred at 25° C. for 8 hours. After the reaction was completed, the solvent was directly removed by rotary evaporation, and the crude product was purified by column chromatography (petroleum ether:ethyl acetate=10:1-1:2) to obtain compound 006-4. LCMS: (ESI) m/z: 636.0 [M+1]⁺.

### Step 4: Synthesis of Compound 006-5

The compound 006-4 (0.33 g, 518.43 μmol, 1 eq) was dissolved in 1,4-dioxane (2 mL), and hydrochloric acid (12 M, 2 mL, 92.59 eq) was added, and the mixture was reacted at 70° C. for 50 hours. After the reaction was completed, the solvent was directly removed by rotary evaporation to obtain a hydrochloride of compound 006-5. LCMS: (ESI) m/z: 480.9 [M+1]⁺.

### Step 5: Synthesis of Compound 006

The hydrochloride salt of compound 006-5 (100 mg, 207.75 μmol, 1 eq) and (E)-4-fluorobut-2-enoic acid (21.62 mg, 207.75 μmol, 1 eq) were dissolved in N,N-dimethylformamide (1 mL), and triethylamine (3.07 mg, 623.25 μmol, 86.75 μL, 3 eq) and propylphosphonic anhydride (264.41 mg, 415.50 μmol, 247.11 μL, 2 eq) were subsequently added. The mixture was stirred at 25° C. for 4 hours. After the reaction was completed, the reaction solution was concentrated, and the crude product was carried out preparative separation, chromatographic column: Phenomenex C18 80*40 mm*3 μm; mobile phase: [water (ammonia)-acetonitrile]; acetonitrile %: 36%-66%, 8 min, to obtain compound 006. LCMS:(ESI) m/z:567.0[M+1]⁺; ¹H NMR (400 MHz, METHANOL-d4) δ ppm 1.57-1.78 (m, 1 H) 1.94-2.14 (m, 2 H) 2.47-2.63 (m, 1 H) 3.49-3.58 (m, 0.5 H) 3.92-4.00 (m, 0.5 H) 4.16-4.41 (m, 2 H) 4.58-4.77 (m, 1 H) 4.89-4.96 (m, 1 H) 4.98-5.09 (m, 1 H) 5.09-5.21 (m, 1 H) 6.70-6.81 (m, 1 H) 6.83-6.92 (m, 1 H) 6.93-7.01 (m, 1 H) 7.10 (d, J=8.8 Hz, 1 H) 7.36 (d, J=8.8 Hz, 2 H) 7.55 (br d, J=8.0 Hz, 2 H) 7.81 (d, J=6.0 Hz, 1 H) 8.04 (dd, J=8.8, 2.5 Hz, 1 H) 8.21-8.27 (m, 1 H).

### EXAMPLE 7

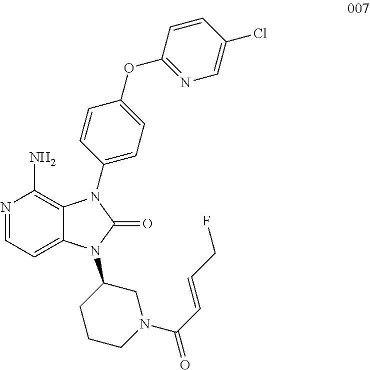 [SMILES: Nc1nccc2c1n(-c1ccc(Oc3ccc(Cl)cn3)cc1)c(=O)n2[C@@H]1CCCN(C(=O)/C=C/CF)C1]

### Synthetic Route

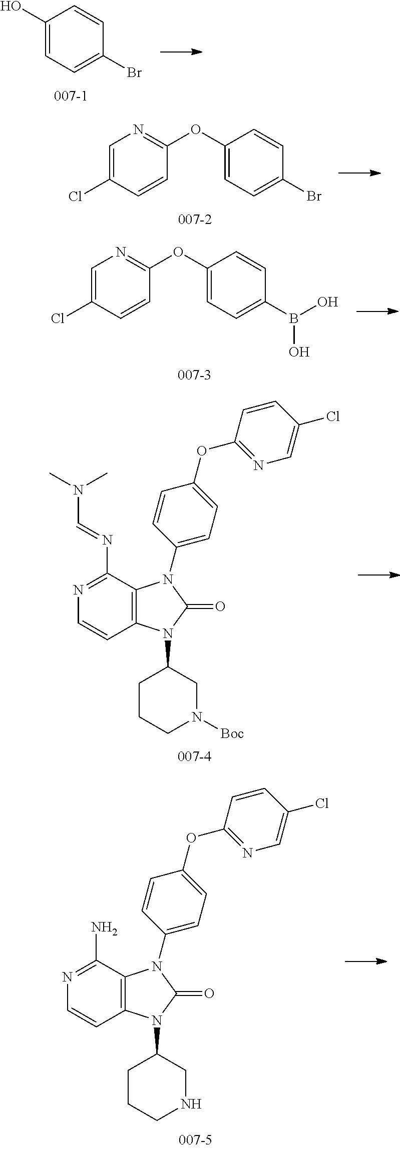 [SMILES: Oc1ccc(Br)cc1 ; Clc1ccc(Oc2ccc(Br)cc2)nc1 ; OB(O)c1ccc(Oc2ccc(Cl)cn2)cc1 ; CN(C)/C=N/c1nccc2c1n(-c1ccc(Oc3ccc(Cl)cn3)cc1)c(=O)n2[C@@H]1CCCN(C(=O)OC(C)(C)C)C1 ; Nc1nccc2c1n(-c1ccc(Oc3ccc(Cl)cn3)cc1)c(=O)n2[C@@H]1CCCNC1]

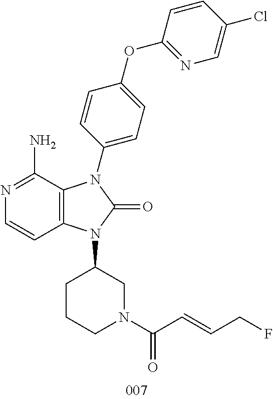 [SMILES: Nc1nccc2c1n(-c1ccc(Oc3ccc(Cl)cn3)cc1)c(=O)n2[C@@H]1CCCN(C(=O)/C=C/CF)C1]

### Step 1: Synthesis of Compound 007-2

The compound 007-1 (4 g, 23.12 mmol, 1 eq) and 5-chloro-2-fluoropyridine (3.04 g, 23.12 mmol, 1 eq) were dissolved in N,N-dimethylformamide (40 mL), and cesium carbonate (15.07 g, 46.24 mmol, 2 eq) was added. The mixture was reacted at 110° C. for 4 hours. After the reaction was complete, it was carried out suction filtration to remove solid impurities, and the filtrate was washed with water and extracted with ethyl acetate twice, 50 mL each time. The organic phases were combined and dried over anhydrous sodium sulfate, and the crude product was purified by column chromatography (petroleum ether: ethyl acetate =10:1) to obtain compound 007-2. LCMS: (ESI) m/z: 283.7 [M+1]⁺.

### Step 2: Synthesis of Compound 007-3

The compound 007-2 (0.3 g, 1.54 mmol, 1 eq) was dissolved in tetrahydrofuran (18 mL), and n-butyllithium (2.5 M, 632 μL, 1.5 eq) was added at −78° C., and the mixture was stirred for 1 hour after the addition. Triisopropyl borate (0.39 g, 2.18 mmol, 2.42 mL, 2 eq) was added and reacting for 1 hour. After the reaction was completed, water was added to quench the reaction, and the tetrahydrofuran was removed by rotary evaporation. The pH of the aqueous phase was adjusted to 5-6 with dilute hydrochloric acid, and the mixture was extracted with dichloromethane three times, 50 mL each time. The organic phases were combined and dried over anhydrous sodium sulfate, and the solvent was removed by rotary evaporation. The crude product was purified through a silica gel column (petroleum ether:ethyl acetate=10:1-1:2) to obtain compound 007-3. LCMS: (ESI) m/z: 249.8 [M+1]⁺.

### Step 3: Synthesis of Compound 007-4

Copper acetate (36.41 mg, 200.43 μmol, 0.5 eq) and 2,2,6,6-tetramethylpiperidinooxy (75.65 mg, 481.04 μmol, 1.2 eq) were dissolved in dichloromethane (2 mL), and triethylamine (162.25 mg, 1.60 mmol, 223.18 μL, 4 eq), 007-3 (155.72 mg, 400.87 μmol, 1 eq) and 001-8 (0.15 g, 601.30 μmol, 1.5 eq) were added, under oxygen protection, the mixture was stirred at 25° C. for 8 hours. After the reaction was completed, the solvent was directly removed by rotary evaporation, and the crude product was purified by column chromatography (petroleum ether: ethyl acetate =10:1-1:2) to obtain compound 007-4. LCMS: (ESI) m/z: 592.0 [M+1]⁺.

### Step 4: Synthesis of Compound 007-5

The compound 007-4 (80 mg, 135.12 μmol, 1 eq) was dissolved in 1,4-dioxane (2 mL), and hydrochloric acid (12 M, 2 mL, 92.59 eq) was added, and the mixture was stirred at 70° C. for 50 hours. After the reaction was completed, the solvent was directly removed by rotary evaporation to obtain a hydrochloride of compound 007-5. LCMS: (ESI) m/z: 436.9 [M+1]⁺.

### Step 5: Synthesis of Compound 007

The hydrochloride salt of compound 007-5 (60.00 mg, 137.33 μmol, 1 eq) and (E)-4-fluorobut-2-enoic acid (14.29 mg, 137.33 μmol, 1 eq) were dissolved in N,N-dimethylformamide (2 mL), and triethylamine (41.69 mg, 412.00 μmol, 57.35 μL, 3 eq) and propylphosphonic anhydride (174.79 mg, 274.67 μmol, 163.35 μL, 50% content, 2 eq) were subsequently added. The mixture was stirred at 25° C. for 4 hours. After the reaction was completed, the reaction solution was concentrated, and the crude product was carried out preparative separation: chromatographic column: Phenomenex C18 80*40 mm*3 μm; mobile phase: [water (ammonia)-acetonitrile]; acetonitrile %: 35%-65%, 8 min, to obtain compound 007. LCMS: (ESI) m/z: 523.1 [M+1]⁺.

### EXAMPLE 8

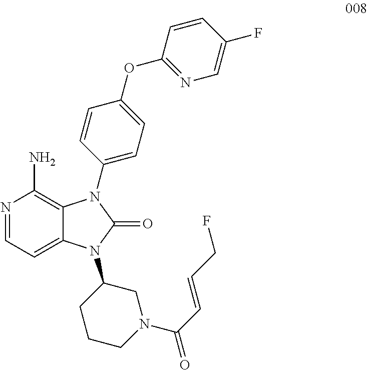 [SMILES: Nc1nccc2c1n(-c1ccc(Oc3ccc(F)cn3)cc1)c(=O)n2[C@@H]1CCCN(C(=O)/C=C/CF)C1]

### Synthetic Route

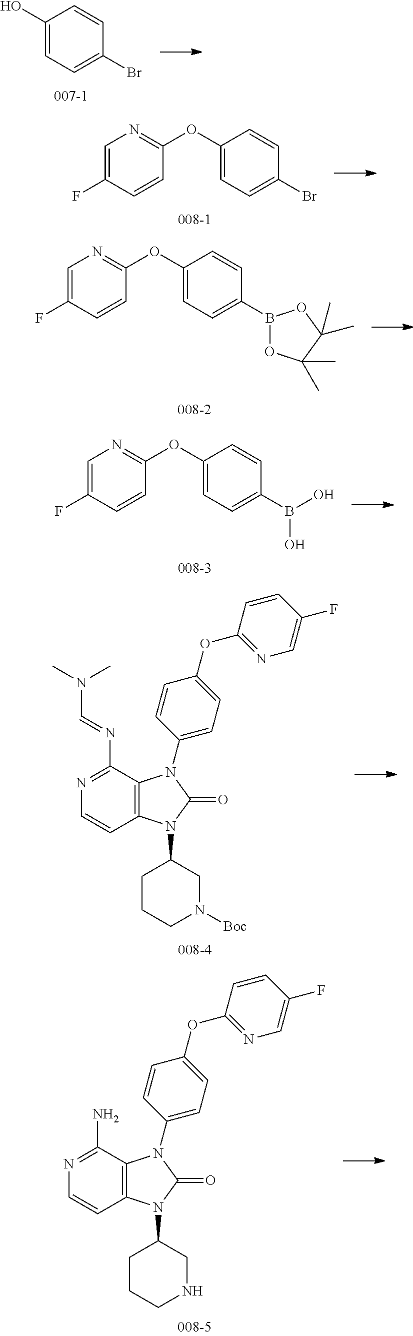

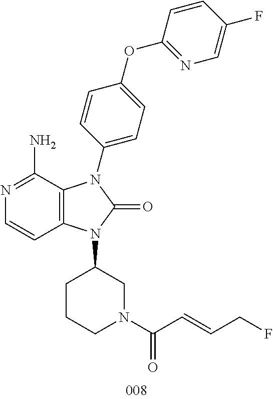 [SMILES: Nc1nccc2c1n(-c1ccc(Oc3ccc(F)cn3)cc1)c(=O)n2[C@@H]1CCCN(C(=O)/C=C/CF)C1]

### Step 1: Synthesis of Compound 008-1

The compound 007-1 (4 g, 23.12 mmol, 1 eq), 2,5-difluoropyridine (2.66 g, 23.12 mmol, 1 L, 1 eq) were dissolved in N,N-dimethylformamide (40 mL), and cesium carbonate (15.07 g, 46.24 mmol, 2 eq) was added, and the mixture was reacted at 80° C. for 4 hours. After the reaction was completed, it was carried out suction filtration, and water was added to the filtrate. The mixture was extracted with ethyl acetate twice, 50 mL each time, washed with saturated sodium chloride. The organic phases were combined and dried over anhydrous sodium sulfate. The solvent was removed by rotary evaporation, and the crude product was purified by a silica gel column (petroleum ether:ethyl acetate=10:1-5:1) to obtain compound 008-1. LCMS:(ESI) m/z:267.6 [M+1]⁺; ¹H NMR (400 MHz, CDCl₃) δ ppm 6.95 (dd, J=9.03, 3.51 Hz, 1 H) 7.03 (d, J=9.03 Hz, 2 H) 7.47 (ddd, J=9.10, 7.34, 3.14 Hz, 1 H) 7.52 (d, J=8.78 Hz, 2 H) 8.04 (d, J=3.26 Hz, 1 H).

### Step 2: Synthesis of Compound 008-2

The compound 008-1 (4 g, 14.92 mmol, 1 eq), bis(pinacolato)diboron (4.93 g, 19.40 mmol, 1.3 eq) and [1,1′- bis(diphenylphosphino)ferrocene]dichloropalladium(II) (2.18 g, 2.98 mmol, 0.2 eq) were dissolved in 1,4-dioxane (40 mL), and potassium carbonate (6.19 g, 44.76 mmol, 3 eq) was added, under nitrogen protection, the mixture was reacted at 100° C. for 8 hours. The solvent was directly removed by rotary evaporation after the reaction was completed, and the crude product was purified by column chromatography (petroleum ether 100%-petroleum ether:ethyl acetate=5:1) to obtain compound 008-2. LCMS: (ESI) m/z: 315.9 [M+1]⁺.

### Step 3: Synthesis of Compound 008-3

The compound 008-2 (5.4 g, 17.13 mmol, 1 eq) was dissolved in acetone (80 mL) and water (20 mL), and sodium periodate (10.99 g, 51.40 mmol, 2.85 mL, 3 eq) and ammonium acetate (2.64 g, 34.27 mmol, 2 eq) were added. The mixture was reacted at 25° C. for 24 hours, the acetone was removed by rotary evaporation after the reaction was completed. The pH of the aqueous phase was adjusted to 5-6 with dilute hydrochloric acid. The mixture was extracted with ethyl acetate twice, 50 mL each time, and washed with saturated sodium chloride. The organic phases were combined and dried over anhydrous sodium sulfate. The crude product was purified by column chromatography (petroleum ether:ethyl acetate=5:1-1:2) to obtain compound 008-3. LCMS: (ESI) m/z: 233.8 [M+1]⁺.

### Step 4: Synthesis of Compound 008-4

Copper acetate (58.45 mg, 321.78 μmol, 0.5 eq) and 2,2,6,6-tetramethylpiperidinooxy (121.44 mg, 772.27 μmol, 1.2 eq) were dissolved in dichloromethane (2 mL), and triethylamine (260.49 mg, 2.57 mmol, 358.31 μL, 4 eq), 008-3 (0.25 g, 643.56 μmol, 1 eq) and 001-8 (224.93 mg, 965.34 μmol, 1.5 eq) were added, under oxygen protection, the mixture was stirred at 25° C. for 8 hours. After the reaction was completed, the solvent was directly removed by rotary evaporation, and the crude product was purified by column chromatography (petroleum ether:ethyl acetate=10:1-1:2) to obtain compound 008-4. LCMS: (ESI) m/z: 576.1 [M+1]⁺.

### Step 5: Synthesis of Compound 008-5

The compound 008-4 (0.18 g, 312.70 μmol, 1 eq) was dissolved in 1,4-dioxane (2 mL), and hydrochloric acid (12 M, 26.06 μL, 1 eq) was added, and the mixture was stirred at 70° C. for 50 hours. After the reaction was completed, the solvent was directly removed by rotary evaporation to obtain a crude hydrochloride salt of compound 008-5. LCMS: (ESI) m/z: 421.0 [M+1]⁺.

### Step 6: Synthesis of Compound 008

The hydrochloride salt of compound 008-5 (100 mg, 237.85 μmol, 1 eq) and (I)-4-fluorobut-2-enoic acid (24.75 mg, 237.85 μmol, 1 eq) were dissolved in N,N-dimethylformamide (2 mL), then triethylamine (72.20 mg, 713.54 μmol, 99.32 μL, 3 eq) and propylphosphonic anhydride (302.71 mg, 475.69 μmol, 282.91 μL, 50% content, 2 eq) were added, and the mixture was stirred at 25° C. for 4 hours. After the reaction was completed, the reaction solution was concentrated, and the crude product was carried out preparative separation, chromatographic column: Xtimate C18 150*40 mm*5 μm; mobile phase: [water (formic acid)-acetonitrile]; acetonitrile %: 22%-62%, 8 min, to obtain compound 008. LCMS: (ESI) m/z: 507.0 [M+1]⁺.

### EXAMPLE 9

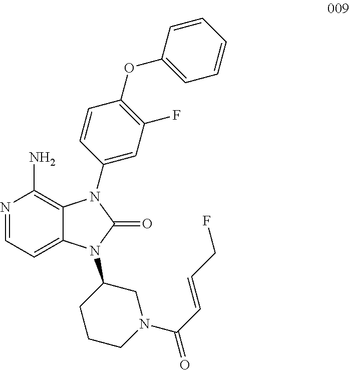 [SMILES: Nc1nccc2c1n(-c1ccc(Oc3ccccc3)c(F)c1)c(=O)n2[C@@H]1CCCN(C(=O)/C=C/CF)C1]

### Synthetic Route

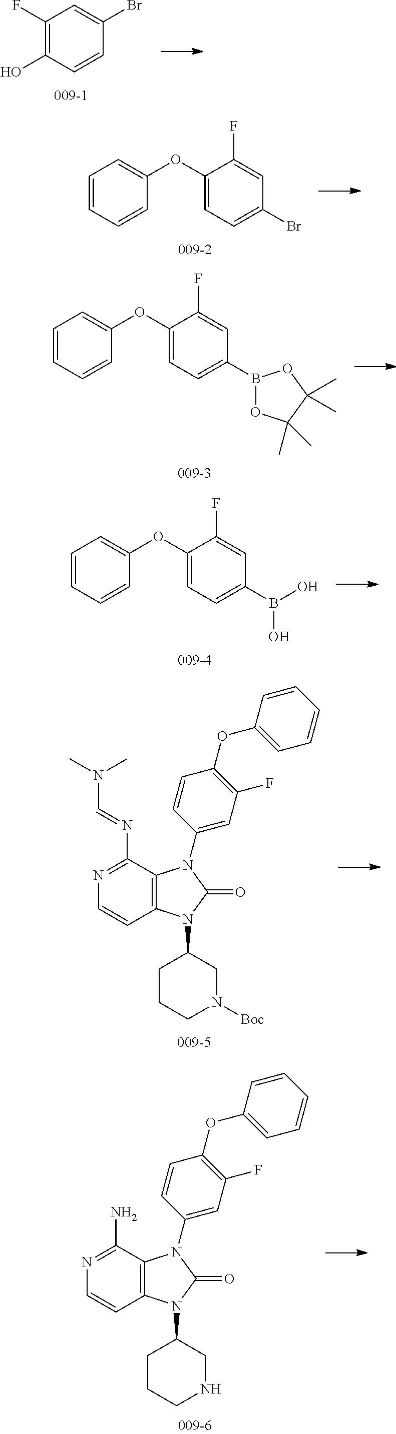

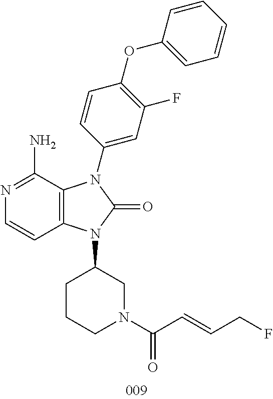 [SMILES: Nc1nccc2c1n(-c1ccc(Oc3ccccc3)c(F)c1)c(=O)n2[C@@H]1CCCN(C(=O)/C=C/CF)C1]

### Step 1: Synthesis of Compound 009-2

Copper acetate (475.49 mg, 2.62 mmol, 0.5 eq) was dissolved in dichloromethane (20 mL), and 1 g of 4A molecular sieve and triethylamine (2.12 g, 20.94 mmol, 2.91 mL, 4 eq) were added, then compound 009-1 (1.00 g, 5.24 mmol, 1 eq) and phenylboron (957.57 mg, 7.85 mmol, 1.5 eq) were added, and oxygen was introduced. The mixture was stirred at 20° C. for 8 hours. After the reaction was completed, it was not treated and was directly removed by rotary evaporation. The crude product was purified by column chromatography (petroleum ether) to obtain compound 009-2. ¹H NMR (400 MHz, CDCl₃) δ ppm 6.92-7.00 (t, J=8.7 Hz, 1 H) 7.01-7.04 (d, J=8.3 Hz, 2 H) 7.1-7.2 (m, 1 H) 7.2-7.3 (m, 1 H) 7.3-7.4 (m, 3 H).

### Step 2: Synthesis of Compound 009-3

The compound 009-2 (1.20 g, 4.49 mmol, 1 eq), bis(pinacolato)diboron (1.48 g, 5.84 mmol, 1.3 eq) and [1,1′- bis(diphenylphosphino)ferrocene]dichloropalladium(II) (657.49 mg, 898.56 μmol, 0.2 eq) were dissolved in 1,4-dioxane (24 mL), and potassium carbonate (1.74 g, 12.58 mmol, 2.8 eq) was added, under nitrogen protection, the mixture was reacted at 100° C. for 8 hours. The solvent was directly removed by rotary evaporation after the reaction was completed, and the crude product was purified by column chromatography (petroleum ether 100%-petroleum ether:ethyl acetate=5:1) to obtain compound 009-3.

### Step 3: Synthesis of Compound 009-4

The compound 009-3 (0.6 g, 1.91 mmol, 1 eq) was dissolved in acetone (20 mL) and water (5 mL), and sodium periodate (1.23 g, 5.73 mmol, 317.49 μL, 3 eq) and ammonium acetate (294.42 mg, 3.82 mmol, 2 eq) were added. The mixture was reacted at 25° C. for 24 hours, and the acetone was removed by rotary evaporation after the reaction was complete. The pH of the aqueous phase was adjusted to 5-6 with dilute hydrochloric acid. The mixture was extracted with ethyl acetate and washed with saturated sodium chloride. The organic phases were combined and dried over anhydrous sodium sulfate, and the crude product was purified by column chromatography (petroleum ether:ethyl acetate=5:1-1:2) to obtain compound 009-4. ¹H NMR (400 MHz, CDCl₃) 8 ppm 7.07-7.13 (m, 3 H) 7.17-7.24 (m, 1 H) 7.38-7.45 (m, 2 H) 7.89 - 8.00 (m, 2 H).

### Step 4: Synthesis of Compound 009-5

The 2,2,6,6-tetramethylpiperidinooxy (64.77 mg, 411.88 μmol, 1.2 eq) and copper acetate (31.17 mg, 171.62 μmol, 0.5 eq) were dissolved in dichloromethane (2 mL), and triethylamine (138.93 mg, 1.37 mmol, 191.10 μL, 4 eq), compound 001-8 (0.20 g, 514.85 μmol, 1.50 eq) and compound 009-4 (119.45 mg, 514.85 μmol, 1.5 eq) were subsequently added, and oxygen was introduced. The mixture was stirred at 25° C. for 12 hours. After the reaction was completed, the reaction solution was concentrated, and the crude product was purified by column chromatography (petroleum ether:ethyl acetate=10%-0.5%) to obtain compound 009-5. LCMS: (ESI) m/z: 575.6 [M+1]⁺.

### Step 5: Synthesis of Compound 009-6

The compound 009-5 (0.15 g, 261.03 μmol, 1 eq) was dissolved in 1,4-dioxane (2 mL), then hydrochloric acid (12 M, 21.75 μL, 1 eq) was added, and the mixture was stirred at 70° C. for 50 hours. After the reaction was completed, the solvent was removed by rotary evaporation to obtain a crude hydrochloride salt of compound 009-6. LCMS: (ESI) m/z: 419.9 [M+1]⁺.

### Step 6: Synthesis of Compound 009

The hydrochloride salt of compound 009-6 (99.76 mg, 237.85 μmol, 1 eq) and (E)-4-fluorobut-2-enoic acid (24.75 mg, 237.85 μmol, 1 eq) were dissolved in N,N-dimethylformamide (2 mL), then triethylamine (72.20 mg, 713.54 μmol, 99.32 μL, 3 eq) and propylphosphonic anhydride (302.71 mg, 475.69 μmol, 282.91 μL, 2 eq) were added, and the mixture was stirred at 25° C. for 4 hours. After the reaction was completed, the reaction solution was concentrated. The crude product was carried out preparative separation, chromatographic column: Xtimate C18 150*40 mm*5 μm; mobile phase: [water (formic acid)-acetonitrile]; acetonitrile %: 26%-66%, 8 min, to obtain compound 009. LCMS: (ESI) m/z: 506.2[M+1]+; ¹H NMR (400 MHz, CDCl₃) 8 ppm 1.−9-1.81 (m, 1 H) 1.−6-2.09 (m, 1 H) 2.21 (br d, J=3.8 Hz, 1 H) 2.−6-2.74 (m, 1 H) 3.−1-3.27 (m, 0.5 H) 3.−3-3.55 (m, 0.5 H) 3.−2-4.00 (m, 1 H) 4.−9-4.16 (m, 2 H) 4.−7-4.90 (m, 1 H) 4.−9-5.10 (m, 1 H) 5.−1-5.22 (m, 1 H) 6.−3-6.66 (m, 1 H) 6.−8-6.81 (m, 1 H) 6.−9-7.03 (m, 1 H) 7.−5-7.26 (m, 6 H) 7.50 (br d, J=3.0 Hz, 3 H).

### EXAMPLE 10

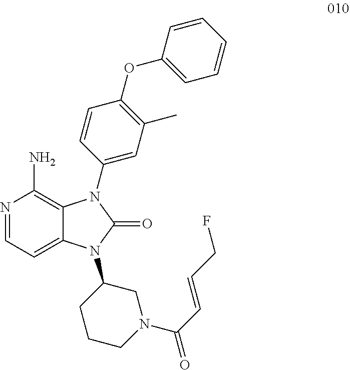 [SMILES: Cc1cc(-n2c(=O)n([C@@H]3CCCN(C(=O)/C=C/CF)C3)c3ccnc(N)c32)ccc1Oc1ccccc1]

### Synthetic Route

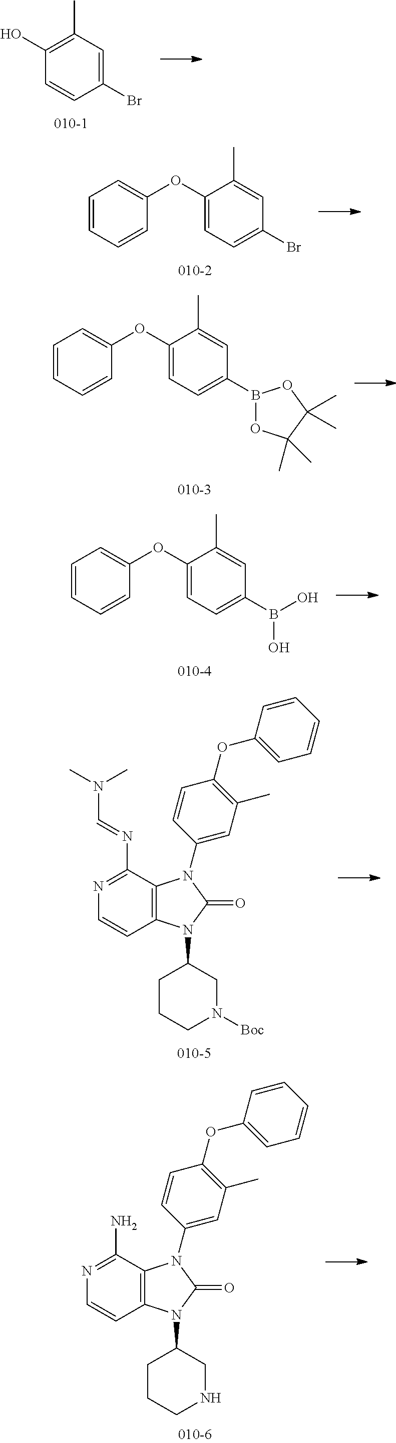

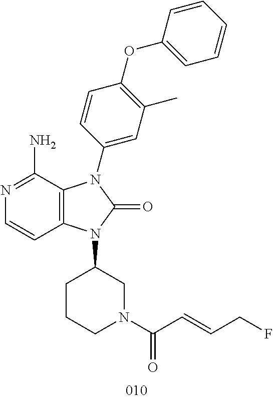 [SMILES: Cc1cc(-n2c(=O)n([C@@H]3CCCN(C(=O)/C=C/CF)C3)c3ccnc(N)c32)ccc1Oc1ccccc1]

### Step 1: Synthesis of Compound 010-2

Copper acetate (485.55 mg, 2.67 mmol, 0.5 eq) was dissolved in dichloromethane (20 mL), and 1 g of 4A molecular sieve and triethylamine (2.16 g, 21.39 mmol, 2.98 mL, 4 eq) were added, then 010-1 (1.00 g, 5.35 mmol, 1 eq) and phenylboronic acid (977.87 mg, 8.02 mmol, 1.5 eq) were added, and oxygen was introduced. The mixture was stirred at 20° C. for 10 hours. After the reaction was completed, it was concentrated, and the crude product was purified by column chromatography (petroleum ether) to obtain compound 010-2.

### Step 2: Synthesis of Compound 010-3

The compound 010-2 (2.86 g, 10.86 mmol, 1 eq), bis(pinacolato)diboron (3.58 g, 14.11 mmol, 1.3 eq), [1,1′-bis(diphenylphosphino)ferrocene]dichloropalladium(II) (1.59 g, 2.17 mmol, 0.2 eq) were added to a 50 mL single-neck flask and dissolved with 1,4-dioxane (40 mL), and potassium carbonate (4.50 g, 32.57 mmol, 3 eq) was added, under nitrogen protection, the mixture was reacted at 100° C. for 8 hours. After the reaction was completed, it was directly concentrated, and the crude product was purified by column chromatography (petroleum ether 100%-petroleum ether:ethyl acetate=5:1) to obtain compound 010-3.

### Step 3: Synthesis of Compound 010-4

The compound 010-3 (1.9 g, 6.13 mmol, 1 eq) was added to a 50 mL single-neck flask and dissolved with acetone (15 mL) and water (3 mL), then sodium periodate (3.93 g, 18.38 mmol, 1.02 mL, 3 eq) and ammonium acetate (944.26 mg, 12.25 mmol, 2 eq) were added, and the mixture was reacted at 25° C. for 24 hours. After the reaction was completed, the acetone was removed by rotary evaporation, and the pH of the aqueous phase was adjusted to 5-6 with dilute hydrochloric acid. The mixture was extracted with ethyl acetate and washed with saturated sodium chloride. The organic phases were combined and dried over anhydrous sodium sulfate. The solvent was removed by rotary evaporation and the crude product was purified by column chromatography (petroleum ether:ethyl acetate=5:1-1:2) to obtain compound 010-4. ¹H NMR (400 MHz, CDCl₃) δ ppm 2.31 (s, 3 H) 6.85-6.90 (m, 1 H) 6.93 (dd, J=7.8, 1.0 Hz, 2 H) 7.01-7.08 (m, 1 H) 7.24-7.32 (m, 2 H) 7.92-7.97 (m, 1 H) 8.00-8.05 (m, 1 H).

### Step 4: Synthesis of Compound 010-5

The compound 001-8 (0.2 g, 514.85 μmol, 1.50 eq), 010-4 (117.41 mg, 514.85 μmol, 1.5 eq), 2,2,6,6-tetramethylpiperidinooxy (64.77 mg, 411.88 μmol, 1.2 eq) and copper acetate (31.17 mg, 171.62 μmol, 0.5 eq) were dissolved in dichloromethane (10 mL), then triethylamine (138.93 mg, 1.37 mmol, 191.10 μL, 4 eq) was added, and oxygen was introduced. The mixture was stirred at 25° C. for 15 hours. After the reaction was completed, the solvent was removed by rotary evaporation, and the crude product was purified by a silica gel column (petroleum ether:ethyl acetate=10:1-1:2) to obtain compound 010-5. LCMS: (ESI) m/z: 571.1 [M+1]⁺.

### Step 5: Synthesis of Compound 010-6

The compound 010-5 (0.15 g, 262.84 μmol, 1 eq) was dissolved in 1,4-dioxane (2 mL), then hydrochloric acid (12 M, 21.90 μL, 1 eq) was added, and the mixture stirred at 70° C. for 50 hours. After the reaction was completed, the solvent was directly removed by rotary evaporation to obtain a crude hydrochloride salt of compound 010-6. LCMS: (ESI) m/z: 416.0 [M+1]⁺.

### Step 6: Synthesis of Compound 010

The hydrochloride salt of compound 010-6 (98.82 mg, 237.85 μmol, 1 eq) and (E)-4-fluorobut-2-enoic acid (24.75 mg, 237.85 μmol, 1 eq) were dissolved in N,N-dimethylformamide (2 mL), and triethylamine (72.20 mg, 713.54 μmol, 99.32 μL, 3 eq) and propylphosphonic anhydride (302.71 mg, 475.69 μmol, 282.91 μL, 2 eq) were subsequently added. After the mixture was stirred at 25° C. for 4 hours, the reaction solution was concentrated, and the crude product was carried out preparative separation, chromatographic column: Xtimate C18 100*30 mm*3 μm; mobile phase: [water (formic acid)-acetonitrile]; acetonitrile %: 10%-50%, 8min, to obtain compound 010. LCMS: (ESI) m/z: 502.2[M+1]⁺; ¹H NMR (400 MHz, CDCl₃) δ ppm 1.48-1.70 (m, 1 H) 1.88-1.96 (m, 1 H) 2.04 (m, 1 H) 2.29 (s, 3 H) 2.47-2.70 (m, 1 H) 3.00-3.17 (m, 0.5 H) 3.30-3.48 (m, 0.5 H) 3.53-3.74 (m, 1 H) 3.99-4.10 (m, 2 H) 4.64-4.83 (m, 1 H) 4.89-5.16 (m, 2 H) 6.42-6.61 (m, 1 H) 6.62-6.74 (m, 1 H) 6.79-6.92 (m, 2 H) 6.96 (br d, J=8.0 Hz, 2 H) 7.07-7.16 (m, 2 H) 7.23-7.39 (m, 4 H).

### EXAMPLE 11

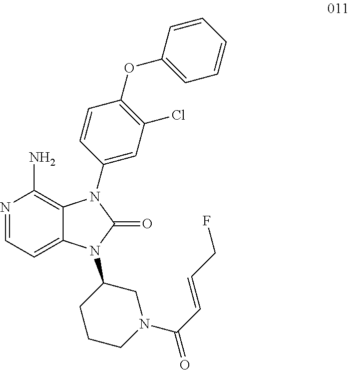 [SMILES: Nc1nccc2c1n(-c1ccc(Oc3ccccc3)c(Cl)c1)c(=O)n2[C@@H]1CCCN(C(=O)/C=C/CF)C1]

### Synthetic Route

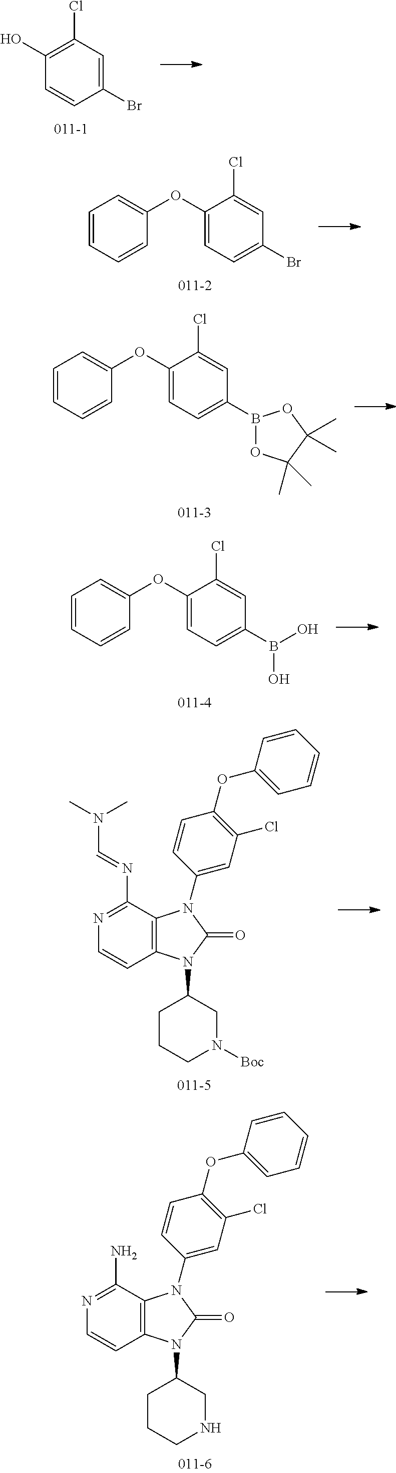

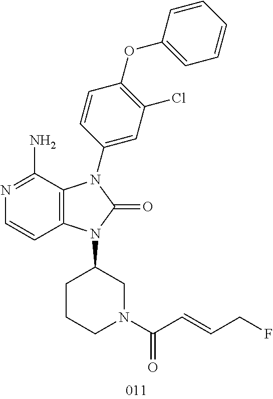 [SMILES: Nc1nccc2c1n(-c1ccc(Oc3ccccc3)c(Cl)c1)c(=O)n2[C@@H]1CCCN(C(=O)/C=C/CF)C1]

### Step 1: Synthesis of Compound 011-2

Copper acetate (2.19 g, 12.05 mmol, 0.5 eq) was added to dichloromethane (40 mL), then 1 g of 4A molecular sieve and triethylamine (9.76 g, 96.41 mmol, 13.42 mL, 4 eq) were added, then 011 -1 (5 g, 24.10 mmol, 1 eq) and phenylboronic acid (4.41 g, 36.15 mmol, 1.5 eq) were added, under oxygen protection, the mixture was stirred at 20° C. for 8 hours. After the reaction was completed, the reaction solution was concentrated, and the crude product was purified by column chromatography (petroleum ether) to obtain compound 011-2. ¹H NMR (400 MHz, CDCl₃) 8 ppm 6.97 (d, J=8.0 Hz, 1 H) 7.00-7.05 (m, 2 H) 7.11-7.18 (m, 1 H) 7.34-7.41 (m, 2 H) 8.00-8.06 (m, 1 H) 8.09-8.14 (m, 1 H).

### Step 2: Synthesis of Compound 011-3

The compound 011-2 (3.2 g, 11.29 mmol, 1 eq), bis(pinacolato)diboron (3.73 g, 14.67 mmol, 1.3 eq) and [1,1′- bis(diphenylphosphino)ferrocene]dichloropalladium(II) (1.65 g, 2.26 mmol, 0.2 eq) was added to a 50 mL single-neck flask, and dissolved in 1,4-dioxane (40 mL) after addition, then potassium carbonate (4.68 g, 33.86 mmol, 3 eq) was added, under nitrogen protection, the mixture was reacted at 100° C. for 8 hours. After the reaction was completed, the reaction solution was directly removed by rotary evaporation, and the crude product was purified by column chromatography (petroleum ether 100%-petroleum ether:ethyl acetate=5:1) to obtain compound 011-3.

### Step 3: Synthesis of Compound 011-4

The compound 011-3 (2 g, 6.05 mmol, 1 eq) was added to a 50 mL single-neck flask and dissolved with acetone (15 mL) and water (3 mL), then sodium periodate (3.88 g, 18.15 mmol, 1.01 mL, 3 eq) and ammonium acetate (932.57 mg, 12.10 mmol, 2 eq) were added, and the mixture was reacted at 25° C. for 24 hours after the addition. The acetone was removed by rotary evaporation, and the pH of the aqueous phase was adjusted to 5-6 with dilute hydrochloric acid. The mixture was extracted with ethyl acetate and washed with saturated sodium chloride. The organic phases were combined and dried over anhydrous sodium sulfate, and the crude product was separated by column chromatography (petroleum ether:ethyl acetate)=5:1-1:2) to obtained compound 011-4. 1H NMR (400 MHz, CDCl₃) δ ppm 6.88-6.95 (m, 1 H) 6.97-7.01 (m, 2 H) 7.08-7.15 (m, 1 H) 7.29-7.35 (m, 2 H) 7.89-7.96 (m, 1 H) 8.13-8.20 (m, 1 H).

Step 4: Synthesis of Compound 011-5

The compound 001-8 (0.2 g, 514.85 μmol, 1.50 eq), 011-4 (127.92 mg, 514.85 μmol, 1.5 eq), 2,2,6,6-tetramethylpiperidinooxy (64.77 mg, 411.88 μmol, 1.2 eq) and copper acetate (31.17 mg, 171.62 μmol, 0.5 eq) were dissolved in dichloromethane (4 mL), then triethylamine (138.93 mg, 1.37 mmol, 191.10 μL, 4 eq) was added, and oxygen was introduced after the addition. The mixture was stirred at 25° C. for 15 hours. After the reaction was completed, the solvent was removed by rotary evaporation, and the crude product was separated by column chromatography (petroleum ether:ethyl acetate=10:1-1:2) to obtain compound 011-5.

### Step 5: Synthesis of Compound 011-6

The compound 011-5 (0.15 g, 253.76 μmol, 1 eq) was dissolved in 1,4-dioxane (2 mL), then HCl (12 M, 21.90 μL, 1 eq) was added, and the mixture was stirred at 70° C. for 50 h. After the reaction was completed, the solvent was directly removed by rotary evaporation to obtain a hydrochloride of crude compound 011-6. LCMS: (ESI) m/z: 435.9 [M+1]⁺.

### Step 6: Synthesis of Compound 011

The hydrochloride salt of compound 011-6 (0.1 g, 229.41 μmol, 1 eq) and (I)-4-fluorobut-2-enoic acid (23.88 mg, 229.41 μmol, 1 eq) were dissolved in N,N-dimethylformamide (2 mL), then triethylamine (69.64 mg, 688.22 μmol, 95.79 μL, 3 eq) and propylphosphonic anhydride (291.97 mg, 458.82 μmol, 272.87 μL, 50% content, 2 eq) were subsequently added, and the mixture was stirred at 25ºC for 12 hours. After the reaction was completed, the reaction solution was concentrated, and the crude product was carried out preparative separation, chromatographic column: Xtimate C18 150*40 mm*5 μm; mobile phase: [water (formic acid)-acetonitrile]; acetonitrile%: 26%-66%, 8min, to obtain compound 011. LCMS: (ESI) m/z: 522.0[M+1]⁺; ¹H NMR (400 MHz, CDCl₃) δ ppm 1.26-1.41 (m, 1 H) 1.55-1.80 (m, 1 H) 1.92-2.22 (m, 2 H) 2.53-2.76 (m, 1 H) 3.09-3.30 (m, 0.5 H) 3.42-3.55 (m, 0.5 H) 4.09-4.20 (m, 2 H) 4.77-4.94 (m, 1 H) 5.00-5.20 (m, 2 H) 6.49-6.72 (m, 1 H) 6.72-6.86 (m, 1 H) 6.88-7.00 (m, 1 H) 7.04(d, J=8.8 Hz, 1 H) 7.11 (d, J=7.8 Hz, 2 H) 7.22-7.31 (m, 3 H) 7.40-7.49 (m, 2 H) 7.59-7.65 (m, 1 H).

### Experimental Example 1: BTK Kinase Experiment

### Experimental Materials:

BTK kinase, PolyE4Y1 substrate and Kinase assay buffer III were purchased from Signalchem; ADP-Glo Kinase Assay was purchased from Promega; Nivo multi-label analyzer (PerkinElmer).

### Experimental Methods:

1× buffer preparation (which was prepared and used immediately): Kinase assay buffer III was diluted with ddH2O to 1× assay buffer for later use.

The compound to be tested was diluted with 100% DMSO to 10 μM as the first concentration, and followed by a 5-fold dilution to the 8th concentration using a multi-channel pipette, i.e., it was diluted from 10 μM to 0.128 nM.

Each gradient of the compound to be tested was diluted into a working solution with 5% DMSO using 1× buffer, 1 μL/well was added to the corresponding well, and a double-replicate well experiment was set up. 2 μL of BTK enzyme (4 ng) was add to each well and incubating at 25° C. for 30 minutes. After the incubation was completed, 2 μL of a mixture of substrate and ATP (2 μM ATP, 0.2 μg/μL PolyE4Y1) was added to each well and incubating at 25° C. for 120 minutes. At this point, the final concentration gradient of the compound was diluted from 100 nM to 0.00128 nM. After the reaction was completed, 5 μL of ADP-Glo reagent was added to each well, and the reaction continued at 25° C. for 40 minutes. After the reaction was completed, 10 μL of kinase detection reagent was added to each well. After reacting at 25° C. for 30 minutes, PerkinElmer Nivo multi-label analyzer was used to read the chemiluminescence, and the integration time was 0.5 seconds.

### Data Analysis:

Raw data was converted into inhibition rate using the equation: (Sample−Min)/(Max−Min)*100%. The IC₅₀ value could then be derived by performing a curve fit using four-parameter (which was obtained using the “log(inhibitor) vs. response-Variable slope” mode in GraphPad Prism). Table 1 provides the effects of the compounds of the present invention on BTK enzyme activity.

TABLE 1
BTK in vitro activity test results

| Compound No. | BTK IC₅₀ (nM) |
|---|---|
| 001 | 0.71 |
| 002 | 1.65 |
| 003A | 4.4 |
| 003B | 1.9 |
| 004 | 2.46 |
| 005 | 2.85 |
| 006 | 13.61 |
| 007 | 16.04 |
| 008 | 36.5 |
| 009 | 2.8 |
| 010 | 3.04 |
| 011 | 1.57 |

Experimental conclusion: The compound of the present disclosure has a strong inhibitory effect on BTK.

### Experimental Example 2: HPBMC Experiment

### Experimental Procedure

### 1.1 Preparation of Medium and Buffer

1) Medium: RPMI Medium 1640+1% P/S+10% FBS
2) MACS buffer: 2% FBS+1 mM EDTA in DPBS
3) Staining buffer: DPBS+2% FBS
1.2 Dilution of Compounds and Anti-Human IgM

The initial concentration of the compound to be tested was 5 μL, 3-fold dilution, 9 points, and it was added to a 96 well plate after dilution, 50 μL/ well.

The anti-human IgM was diluted according to Table 2 below, and added to the 96 well plate after dilution, 50 μL/well.

TABLE 2
Dilution method of anti-human IgM

| | Concentration | | Dilution |
|---|---|---|---|
| Name | μg/ml | Dilution method | factor |
| Storage solution | 1200 | NA | NA |
| 4* working | 40 | 260 μL stock + | 30 |
| concentration | | 7540 μL CM | |

### 1.3 Preparation and Processing of B Cells

1) PBMC was removed from a liquid nitrogen tank, then resuscitated and thawed in a 37° C. water bath;
2) Using a B cell olation kit, B cells were isolated from PBMC according to the instructions, and their viability and number were calculated using a cell counter.

Table 10 Number and viability of PBMC and B cells
3) The cells were resuspended with 1640 culture medium to a final concentration of 1 M/mL.
4) The B cells were spread at 0.1 M/ well (100 μL) into a 96-well flat-bottom plate already containing the compound and anti-IgM.
5) The plate was incubated in a 37° C. CO2 incubator for 48 hours;

### 1.4 Cell Staining and Flow Cytometry Analysis

1) The cells were collected 48 hours later, and transferred to a 96 V bottom plate, 350 g, and centrifuged for 5 minutes, the supernatant was discarded, and 200 μL of DPBS was added to wash once, the mixture was centrifuged again, and the supernatant was discarded;
2) 50 μL of DPBS and 50 μL of Live/Dead (1:500 PBS dilution) were added to resuspend at room temperature for 10 minutes;
3) 100 μL of the staining buffer was added to wash once, 350 g, centrifuging and the supernatant was discarded;
4) 20 μL of Fc receptor blocking solution was added to each well, the cells were resuspended for blocking at room temperature for 10 minutes; (the Fc receptor blocking solution was diluted 20 times with the staining buffer);
5) 20 μL of APC-labeled mouse anti-human CD69 (20-fold dilution with staining buffer) or isotype control was added to each well, 4° C., 30 min.
6) 150 μL staining buffer was added to washed twice, 350 g, centrifuging for 5 minutes and the supernatant was discarded;
7) 100 μL of the staining buffer was added to resuspend the cells, and the flow cytometer was used.

### 2. Statistical Processing

The data were processed by GraphPad Prism statistical software and represented by Mean+SEM. Activity data were shown in Table 3.

TABLE 3
HPBMC cell activity

| Compound No. | CD69 IC₅₀ (nM) |
|---|---|
| 002 | 3.6 |
| 003B | 2.0 |

Experimental conclusion: this series of compounds had a good inhibitory effect on HPBMC cells.

### Experimental Example 3: Plasma Protein Binding Experiment (PPB)

Experimental operation: 995 μL of blank plasma of each genus was taken, 5 μL of working solution of test compound (400 μM) or working solution of warfarin (400 μM) was added, so that the final concentrations of the test compound and warfarin in the plasma sample was 2 μM. The sample was mixed thoroughly. The final concentration of the organic phase DMSO was 0.5%; 50 μL of plasma samples of the test compound and warfarin were pipetted into the sample receiving plate (three parallels), and the corresponding volume of the corresponding blank plasma or buffer was immediately added so that the final volume of each sample well was 100 μL, with a volume ratio of 1:1 for plasma: dialysis buffer, and then 500 μL of stop solution was added to these samples, and these samples would be used as a T₀ sample for the recovery and stability determination. The To sample was stored at 2-8° C., waiting for subsequent processing with other dialyzed samples; 150 μL of plasma samples of the test compound and warfarin were added to the administration end of each dialysis well, and 150 μL of blank dialysis buffer was added to the corresponding receiving end of the dialysis well. The dialysis plate was then placed in a moist incubator with 5% CO₂, and incubated at 37° C. for 4 hours with shaking at approximately 100 rpm. After dialysis, 50 μL of dialyzed buffer sample and dialyzed plasma sample were pipetted to a new sample receiving plate. A corresponding volume of the corresponding blank plasma or buffer was added to the sample, so that the final volume of each sample well was 100 μL, with a volume ratio of 1:1 for plasma: dialysis buffer. All samples were analyzed by LC/MS/MS after protein precipitation, and the protein binding rate and recovery rate were calculated by the formula: % Unbound=100*F/T, % Bound=100−% Unbound, % Recovery 100*(F+T) T₀ (where F was the peak area ratio of the compound in the dialysate after 4 hours of dialysis; T was the peak area ratio of the compound in the plasma after 4 hours of dialysis, and T₀ was the peak area ratio of the compound in the plasma sample at zero time). The experimental results were shown in Table 4:

TABLE 4
Test results of PPB

| Compound No. | Unbound PPB H/D/C/R/M |
|---|---|
| 002 | 8.2%/4.3%/4.1%/1.3%/4.5% |
| 003B | 10.7%/10.9%/9.5%/5.7%/1.8% |

Experimental conclusion: The compound of the present invention had a strong binding with plasma proteins.

### Experimental Example 4: Hepatocyte Metabolic Stability Experiment (HMS)

Experimental operations: 198 μL of hepatocyte suspension (0.51×106 cells/mL) was added to the preheated incubation plate, and 198 μL of culture medium without hepatocytes was added to T0-MC and T120-MC incubation plates for the culture medium control group, and all incubation plates were pre-incubated for 10 minutes in a 37° C. incubator. Then 2 μL of the working solution of the test and control compounds were added and mixed well, and the incubation plate was immediately put into the plate shaker in the incubator, the rotation speed was adjusted to about 650 rpm, and the timer and the reaction were started. 2 duplicate samples for each compound were prepared at each time point. Incubation conditions: 37° C., saturated humidity, and containing 5% CO2. In the test system, the final concentration of the test compound was 1 μM, the final concentration of the control compound was 3 μM, the final concentration of hepatocytes was 0.5×106 cells/mL, and the final concentration of the total organic solvent was 0.96%, of which the final concentration of DMSO was 0.1%. At the end of the incubation at the corresponding time point, the incubation plate was took out, and 25 μL of the compound and the control compound mixed with the cells was removed and added to the sample plate containing 125 μL of stop solution (acetonitrile-methanol solution (v:v, 5:95) containing 200 ng/mL tolbutamide). For Blank sample plates, 25 μL of culture medium without hepatocytes was added directly. All sample plates were sealed and shaken on a plate shaker at 600 rpm for 10 minutes and then centrifuged at 3220×g for 20 minutes. The supernatant of the test compound was diluted with ultrapure water at a ratio of 1:1, and the supernatant of the control compound was diluted with ultrapure water at a ratio of 1:3. All samples were mixed and analyzed by LC-MS/MS method. The experimental results were shown in Table 5:

TABLE 5
Test results of HMS

| Compound | HMS CLint(liver) (mL/min/kg) |
|---|---|
| No. | H/D/C/R/M |
| 002 | 130.1/186.7/203.1/171/701.6 |
| 003B | 39.8/<44/73/297/2766 |

CLint(liver): hepatic intrinsic clearance rate

Experimental conclusion: This series of compounds had a good hepatocyte stability.

### Experimental Example 5: Evaluation of Compound for Inhibition of Cytochrome P450 Enzyme In Vitro

### Experimental Purpose

To investigate the activity of compounds on human liver microsomal CYP450 enzyme, the compounds were tested for the inhibitory effect of five major subtypes of human hepatocyte CYP450 enzymes (CYP1A2, 2C9, 2C19, 2D6 and 3A4) using LC-MS/MS method. The IC₅₀ value was used as an indicator for compound screening and analysis.

### Experimental Methods and Procedures

1) Working solutions of test compounds and standard inhibitors were prepared (100×);
2) The microsomes were removed from the −80° C. refrigerator, thawed on ice, labeled with the date, and returned to the refrigerator immediately after use;
3) 20 μL of substrate solution was added to the corresponding well;
4) 20 μL of phosphate buffer salt solution was added to the blank well;
5) 2 μL of test compound and positive control working solution were added into the corresponding wells;
6) 2 μL of solvent was added to the wells without the inhibitors and blank wells;
7) Working solution of human liver microsome was prepared;
8) 158 μL of HLM working solution was added to all wells of the incubation plate;
9) The plate was preheated in a 37° C. water bath for about 10 minutes;
10) NADPH cofactor solution was prepared;
11) 20 μL of NADPH cofactor was added to all incubation wells;
12) All CYP was mixed and incubated in a 37° C. water bath for 10 minutes;
13) At the time point, the reaction was terminated by adding 400 μL of cold solution (200 ng/ml acetonitrile solution of tolbutamide and 200 ng/ml acetonitrile solution of Labetalol);
14) The sample was centrifuged at 4000 rpm for 20 minutes to precipitate proteins;
15) 200 μL of supernatant was transferred into 100 μL of high performance liquid chromatography water and shaked well for 10 minutes;
16) LC/MS/MS analysis was started.

The experimental results were shown in Table 6:

TABLE 6
Test results of CYP

| Compound | CYP450 IC₅₀ (μM) |
|---|---|
| No. | 1A2/2C9/2C19/2D6/3A4 |
| 003B | >50/20.7/31.0/>50/>50 |

Experimental conclusion: Compound 003B had no significant inhibitory effect on CYP enzyme.

### Experimental Example 6: Pharmacokinetic Evaluation of Compounds in Mice

Experimental purpose: To test the in vivo pharmacokinetic data of compounds in CD-1 mice.

The compound was mixed with the solvent 5% DMSO/5% Solutol/90% H₂O, vortexed and sonicated to prepare a 0.5 mg/mL clear solution. CD-1 male mice aged 7 to 10 weeks were selected, and administered the candidate compound solution intravenously. Whole blood was collected for a certain period of time, and plasma was prepared. The drug concentration was analyzed by LC-MS/MS method, and pharmacokinetic parameters were calculated by Phoenix WinNonlin software (Pharsight Company, USA). The results were shown in Table 7.

TABLE 7
Intravenous (IV) PK data

| Test Compounds | 002 | 003B |
|---|---|---|
| Dosage of administration (mg/kg) | 1.0 | 1.2 |
| C₀ (nM) | 4192 | 4239 |
| T_{1/2} (h) | 0.28 | 0.27 |
| Vd (L/kg) | 0.76 | 0.85 |
| Cl (mL/Kg/min) | 49 | 79 |
| AUC_{0-inf} (nM · h) | 697 | 445 |

The compound was mixed with the solvent 5% DMSO/5% Solutol/90% H₂O, vortexed and sonicated to prepare a 0.5 mg/mL clear solution. CD-1 male mice aged 7 to 10 weeks were selected, and administered the candidate compound solution orally. Whole blood was collected for a certain period of time, and plasma was prepared. The drug concentration was analyzed by LC-MS/MS method, and pharmacokinetic parameters were calculated by Phoenix WinNonlin software (Pharsight Company, USA). The results were shown in Table 8.

TABLE 8
PK data of Peros (PO)

| Test Compounds | 002 | 003B |
|---|---|---|
| Dosage of administration (mg/kg) | 2.9 | 2.85 |
| Cₘₐₓ (nM) | 1189 | 1004 |
| Tₘₐₓ (h) | 0.083 | 0.083 |
| T_{1/2} (h) | 0.35 | 1.69 |
| AUC_{0-inf} (nM · h) | 830 | 602 |
| F % | 39.7% | 45.1% |

Experimental conclusion: The compounds of the present disclosure had good PK properties in mice.

### Experimental Example 7: Pharmacokinetic Evaluation of Compounds in Rats

Experimental purpose: To test the in vivo pharmacokinetic data of compounds in SD rats.

The compound was mixed with the solvent 5% DMSO/5% Solutol/90% H₂O, vortexed and sonicated to prepare a 0.5 mg/mL clear solution. The rats were administered the candidate compound solution intravenously. Whole blood was collected for a certain period of time, and plasma was prepared. The drug concentration was analyzed by LC-MS/MS method, and pharmacokinetic parameters were calculated. The results were shown in Table 9.

TABLE 9
Intravenous (IV) PK data

| Test Compounds | 002 | 003B |
|---|---|---|
| Dosage of administration (mg/kg) | 1.0 | 0.85 |
| C₀ (nM) | 3515 | 2200 |
| T_{1/2} (h) | 0.40 | 0.32 |
| Vd (L/kg) | 0.7 | 1.16 |
| Cl (mL/Kg/min) | 26.0 | 50.2 |
| AUC_{0-inf} (nM · h) | 1318 | 705 |

The compound was mixed with the solvent 5% DMSO/5% Solutol/90% H₂O, vortexed and sonicated to prepare a 2 mg/mL clear solution. The rats were administered candidate compound solutions orally. Whole blood was collected for a certain period of time, and plasma was prepared. The drug concentration was analyzed by LC-MS/MS method, and pharmacokinetic parameters were calculated. The results were shown in Table 10.

TABLE 10
PK data of Peros (PO)

| Test Compounds | 002 | 003B |
|---|---|---|
| Dosage of administration (mg/kg) | 9.2 | 8.4 |
| Cₘₐₓ (nM) | 1805 | 1125 |
| Tₘₐₓ (h) | 0.38 | 0.38 |
| T_{1/2} (h) | 1.79 | 1.56 |
| AUC_{0-inf} (nM · h) | 2582 | 2117 |
| F % | 18.1% | 30.0% |

Conclusion: The compound of the present invention had good PK properties in rats.

### Experimental Example 8: Pharmacokinetic Evaluation of Compounds in Dogs

Experimental purpose: To test the in vivo pharmacokinetic data of compounds in beagle dogs.

The compound was mixed with the solvent 5% DMSO/5% Solutol/90% H₂O, vortexed and sonicated to prepare a 0.5 mg/mL clear solution. The beagle dogs were administered the candidate compound solution intravenously. Whole blood was collected for a certain period of time, and plasma was prepared. The drug concentration was analyzed by LC-MS/MS method, and pharmacokinetic parameters were calculated. The results were shown in Table 11.

TABLE 11
Intravenous (IV)PK data

| Test Compounds | 002 | 003B |
|---|---|---|
| Dosage of administration | 0.49 | 0.5 |
| (mg/kg) | | |
| C₀ (nM) | 1714 | 451 |
| T_{1/2} (h) | 0.97 | 1.14 |
| Vd (L/kg | 1.50 | 2.49 |
| Cl (mL/Kg/min) | 25.6 | 31.9 |
| AUC_{0-inf} (nM · h) | 684 | 557 |

The compound was mixed with the solvent 5% DMSO/5% Solutol/90% H₂O, vortexed and sonicated to prepare a 0.5 mg/mL clear solution. The beagle dogs were administered candidate compound solutions orally. Whole blood was collected for a certain period of time, and plasma was prepared. The drug concentration was analyzed by LC- MS/MS method, and pharmacokinetic parameters were calculated. The results were shown in Table 12.

TABLE 12
PK data of Peros (PO)

| Test Compounds | 002 | 003B |
|---|---|---|
| Dosage of administration (mg/kg) | 2.0 | 2.0 |
| Cₘₐₓ (nM) | 1135 | 724 |
| Tₘₐₓ (h) | 0.5 | 0.63 |
| T_{1/2} (h) | 0.71 | 1.0 |
| AUC_{0-inf} (nM · h) | 2249 | 2247 |
| F % | 82.2% | 101% |

Conclusion: The compounds of the present invention had good PK properties in dogs.

### Experimental Example 9: In Vivo Efficacy of EAE in C57BL/6 Mice

Experimental methods: Experimental autoimmune encephalomyelitis (EAE) model was established in 10-week-old C57BL/6 female mice, and the animals were randomly divided into two groups according to their weight, of which, group G1 was a pure modeling group with 5 animals, and group G2 was a subject 002 group with 7 animals. On day 0, 100 μL of myelin oligodendrocyte glycoprotein (MOG) emulsion was injected subcutaneously into the hind flank of each animal in the G1-G2 group at two points, totaling 200 μL. After 2 hours and 48 hours, respectively, 200 μL of pertussis toxin (PTX) was injected subcutaneously into the animals of groups G1-G2. The in vivo experiment was finished on day 22, and no treatment was given to group G1 from day 0; G2 group was administered 5 mg/kg of subject 002 once a day for 30 days. The animals in each group were weighed and scored (including tail weakness, claudication, hind limb paralysis, hind limb paralysis and other symptoms) every 2 days.

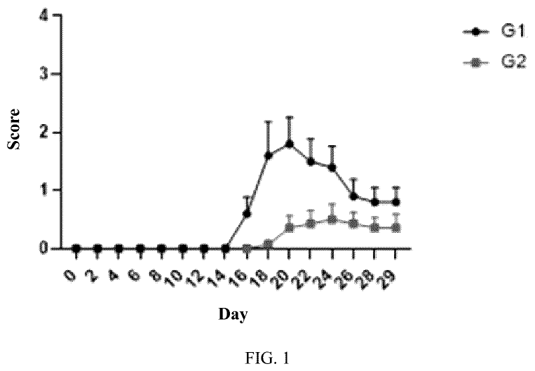

Experimental results: In the experimental autoimmune encephalomyelitis (EAE) model of the C57BL/6 female mice, compound 002 at a dose of 5 mg/kg, QD, was effective in reducing the severity of the disease compared with the modeling group. The scoring criteria were shown in Table 13 and Table 14, and the detailed results were shown in Table 15 and FIG. 1.

TABLE 13
Scoring criteria

| Score | Symptom | Comment |
|---|---|---|
| 0 | Asymptomatic | There were no obvious clinical symptoms and the mouse |
| | | was in normal conditions. When the root of the tail was |
| | | picked up, the mouse's tail was completely erect and could |
| | | move freely. |
| 1 | Tail weakness | When the root of the tail was picked up, the mouse's tail |
| | | was completely hanging down in an inverted “U” shape, |
| | | with no obvious movement response. Both hind limbs |
| | | stretched out, without obvious hind limb symptoms. |
| 2 | Claudication | The mouse's gait was uncoordinated. When the root of |
| | | the tail was picked up, both hind limbs curled up. |
| | | Or, the hind limbs are same as 0, and the single mouse' |
| | | head tilted and unable to maintain balance. |
| 3 | Hind limb | The hind limbs were dragging, and even if there is |
| | paralysis | movement in the paws, both the hind limbs were unable to |
| | | support forward movement, and the mouse relied on the |
| | | forelimbs to move forward. |
| | | Or, single hind limb paralyzed and single forelimb |
| | | paralyzed. |
| | | Or, severe head tilt. |
| | | Or, the mouse leaned against the cage wall. |
| | | Or, When the root of the tail was picked up, the mouse |
| | | rotated and struggled. |
| 4 | Forelimb paralysis | The mouse was basically unable to move and remained |
| | | alert to feeding. |
| 5 | death | — |

Symptoms between two scores was scored as n.5.

TABLE 14
Scoring criteria

| Score | Comment |
|---|---|
| 0.5 | When the root of the tail was picked up, the mouse's tail |
| | could still move, and the tail was in an inverted “J” shape, |
| | with no movement response at the tip of the tail. |
| 1.5 | The mouse was placed on the lid of the iron cage, and the |
| | hind limbs often felled off the lid of the cage, without |
| | obvious claudication. |
| 2.5 | The mouse could still move the hind limbs and some could |
| | still move forward with dragging. |
| | Or, single hind limb was completely dragging. |
| | Or, there were no obvious hind limb symptoms, but the |
| | head tilted and the mouse kept falling. |
| 3.5 | The mouse could still move in the cage, but it was unable |
| | to turn back to its original state to its original shape after |
| | the mouse was turned over. |
| | Or, the hind limbs were completely flat. The forelimbs |
| | arched. |
| 4.5 | The mouse had no obvious movement in the cage and was |
| | not alert to feeding. Dying |

TABLE 15
The efficacy of the subject in autoimmune encephalomyelitis (EAE) model
of mouse

| | Number of | | Frequency of | Dose | Scores of animals on day |
|---|---|---|---|---|---|
| Group | animals | Subject | administration | mg/kg | 20 after administration |
| G1 | 5 | Moulding | QD × 29 | NA | 1.8 |
| | | group | | | |
| G2 | 7 | Compound | QD × 29 | 5 | 0.4 |
| | | 002 | | | |

Experimental conclusion: The compound exhibited good efficacy in the EAE model of mouse.

1. A compound represented by formula (P) or a pharmaceutically acceptable salt thereof, 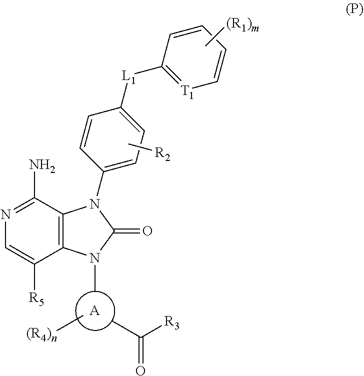
wherein
T₁ is selected from CH and N;
L₁ is selected from O and —C₁₋₃ alkyl-NH—C(═O)—;
ring A is selected from pyrrolidinyl and piperidyl;
each R₁ is independently selected from halogen, C₁₋₃ alkyl and C₁₋₃ alkoxy, respectively, and the C₁₋₃ alkyl and C₁₋₃ alkoxy are independently optionally substituted by 1, 2 or 3 halogens, respectively;
R₂ is selected from H, halogen and C₁₋₃ alkyl, and the C₁₋₃ alkyl is optionally substituted by 1, 2 or 3 halogens;
R₃ is selected from 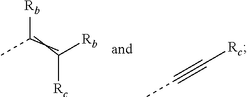
R₄ is selected from halogen, CN, C₁₋₃ alkyl and C₁₋₃ alkoxy, and the C₁₋₃ alkyl and C₁₋₃ alkoxy are independently optionally substituted by 1, 2 or 3 halogens, respectively;
R₅ is selected from H, halogen and C₁₋₃ alkyl, and the C₁₋₃ alkyl is optionally substituted by 1, 2 or 3 halogens;
each R_{b} is independently selected from H and halogen, respectively;
each R_{c} is selected from H and C₁₋₃ alkyl, respectively, and the C₁₋₃ alkyl is substituted by 1, 2 or 3 F;
m is 0, 1, 2 or 3;
n is 0, 1 or 2;
provided that when L₁ is selected from O, R_{c} is selected from C₁₋₃ alkyl, and the C₁₋₃ alkyl is substituted by 1, 2 or 3 F.

2. The compound or the pharmaceutically acceptable salt thereof according to claim 1, wherein L₁ is selected from O and —CH₂—NH—C(═O)—.

3. The compound or the pharmaceutically acceptable salt thereof according to claim 1, wherein each R₁ is independently selected from F, CI, Br, CH₃, CH₂CH₃, CH(CH₃)₂, OCH₃, OCH₂CH₃ and OCH(CH₃)₂, respectively, and the CH₃, CH₂CH₃, CH(CH₃)₂, OCH₃, OCH₂CH₃ and OCH(CH₃)₂ are independently optionally substituted by 1, 2 or 3 halogens, respectively.

4. The compound or the pharmaceutically acceptable salt thereof according to claim 1, wherein each R₁ is independently selected from F, Cl, Br, CH₃, CH₂CH₃, CH(CH₃)₂, CF₃, OCH₃, OCH₂CH₃, OCH(CH₃)₂ and OCF₃, respectively.

5. The compound or the pharmaceutically acceptable salt thereof according to claim 1, wherein the structural moiety 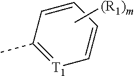
is selected from

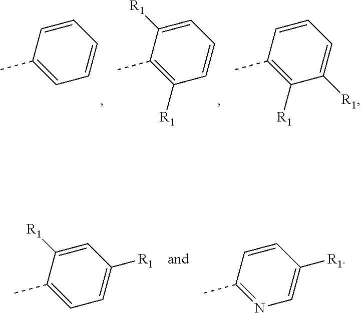

6. The compound or the pharmaceutically acceptable salt thereof according to claim 1, wherein the structural moiety 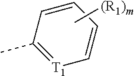
is selected from

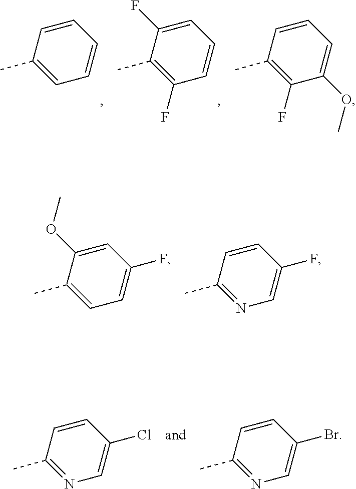

7. The compound or the pharmaceutically acceptable salt thereof according to claim 1, wherein R₂ is selected from H, F, Cl and CH₃; or,
each R_{c} is independently selected from H, CH₃, CH₂CH₃ and CH(CH₃)₂, respectively, and the CH₃, CH₂CH₃ and CH(CH₃)₂ are substituted by 1, 2 or 3 F; or,
R₄ is selected from F, Cl, Br, CN, CH₃, CF₃, OCH₃ and OCF₃; or,
ring A is selected from 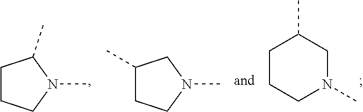
or, R₅ is selected from H.

8. The compound or the pharmaceutically acceptable salt thereof according to claim 1, wherein the structural moiety 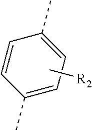
is selected from

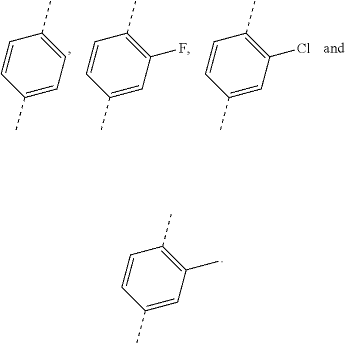

9. (canceled)

10. The compound or the pharmaceutically acceptable salt thereof according to claim 1, wherein each R_{c} is independently selected from H, CH₂F, CHF₂ and CF₃, respectively.

11. The compound or the pharmaceutically acceptable salt thereof according to claim 1, wherein R₃ is selected from

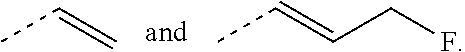

12. (canceled)

13. The compound or the pharmaceutically acceptable salt thereof according to claim 1, wherein R₄ is selected from F and CH₃.

14. (canceled)

15. The compound or the pharmaceutically acceptable salt thereof according to claim 1, wherein, n is 0 or 1.

16. The compound or the pharmaceutically acceptable salt thereof according to claim 1, wherein the structural moiety 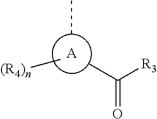
is selected

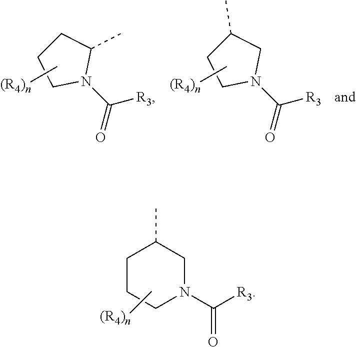

17. The compound or the pharmaceutically acceptable salt thereof according to claim 16, wherein the structural moiety 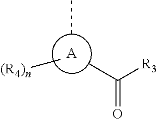
is selected from

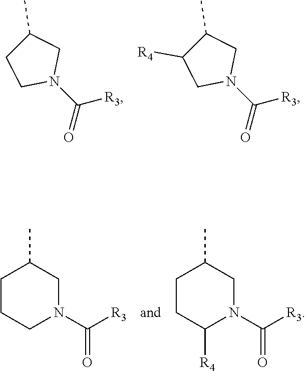

18. The compound or the pharmaceutically acceptable salt thereof according to claim 1, wherein the structural moiety 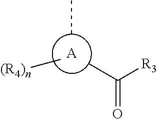
is selected from

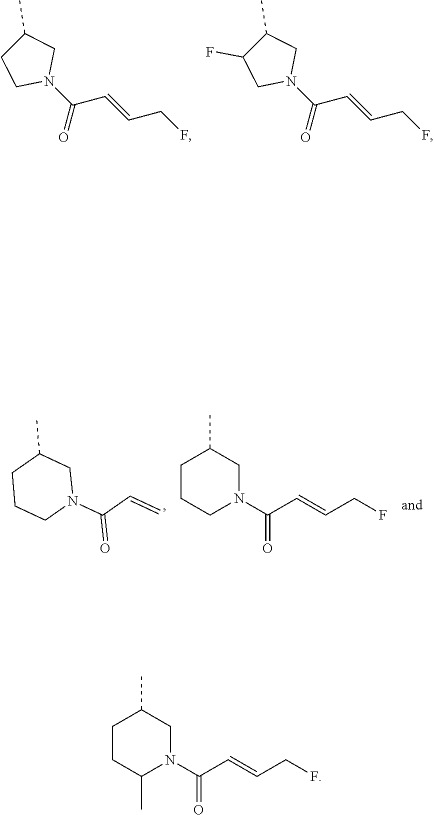

19. (canceled)

20. The compound or the pharmaceutically acceptable salt thereof according to claim 1, the compound is selected from, 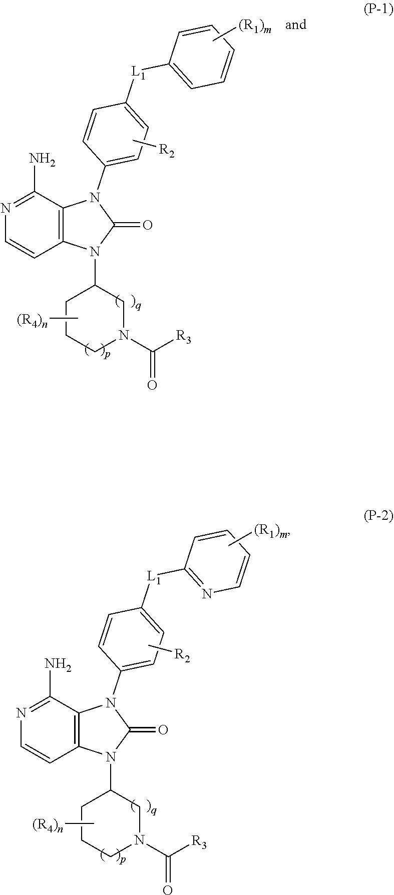
p is 0 or 1, q is 0 or 1, and p and q are not 0 at the same time.

21. A compound represented by the following formula or a pharmaceutically acceptable salt thereof,

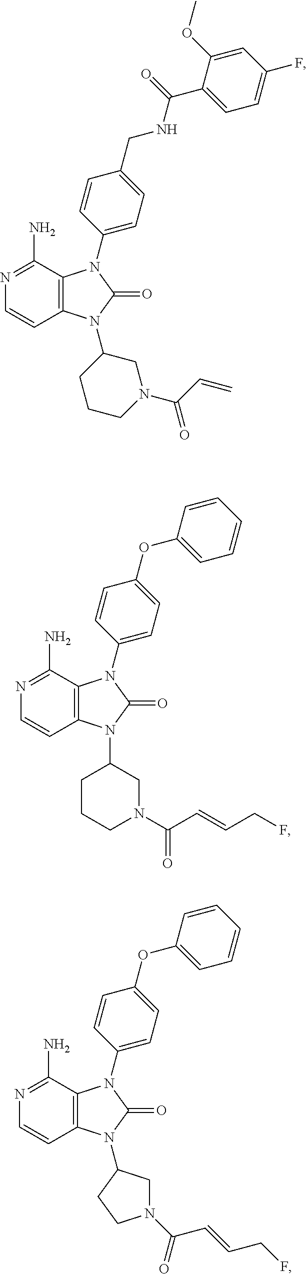 [SMILES: C=CC(=O)N1CCCC(n2c(=O)n(-c3ccc(CNC(=O)c4ccc(F)cc4OC)cc3)c3c(N)nccc32)C1 ; Nc1nccc2c1n(-c1ccc(Oc3ccccc3)cc1)c(=O)n2C1CCCN(C(=O)/C=C/CF)C1 ; Nc1nccc2c1n(-c1ccc(Oc3ccccc3)cc1)c(=O)n2C1CCN(C(=O)/C=C/CF)C1]

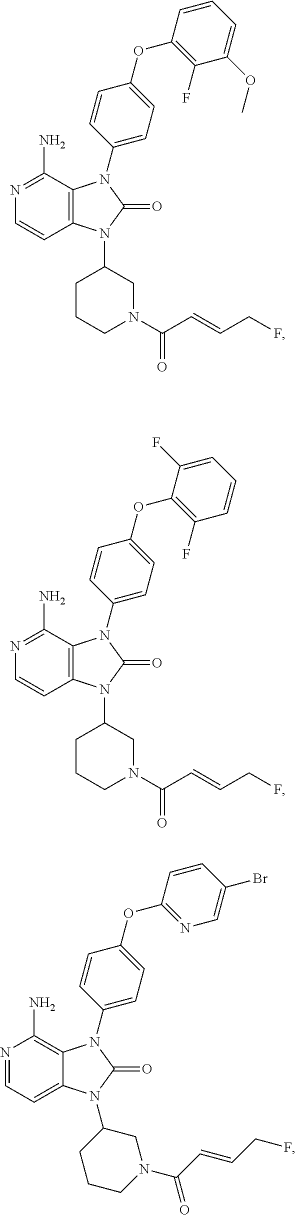 [SMILES: COc1cccc(Oc2ccc(-n3c(=O)n(C4CCCN(C(=O)/C=C/CF)C4)c4ccnc(N)c43)cc2)c1F ; Nc1nccc2c1n(-c1ccc(Oc3c(F)cccc3F)cc1)c(=O)n2C1CCCN(C(=O)/C=C/CF)C1 ; Nc1nccc2c1n(-c1ccc(Oc3ccc(Br)cn3)cc1)c(=O)n2C1CCCN(C(=O)/C=C/CF)C1]

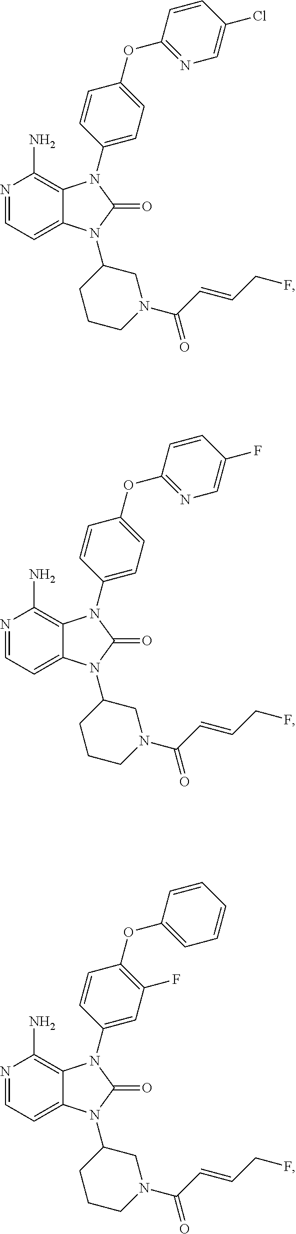 [SMILES: Nc1nccc2c1n(-c1ccc(Oc3ccc(Cl)cn3)cc1)c(=O)n2C1CCCN(C(=O)/C=C/CF)C1 ; Nc1nccc2c1n(-c1ccc(Oc3ccc(F)cn3)cc1)c(=O)n2C1CCCN(C(=O)/C=C/CF)C1 ; Nc1nccc2c1n(-c1ccc(Oc3ccccc3)c(F)c1)c(=O)n2C1CCCN(C(=O)/C=C/CF)C1]

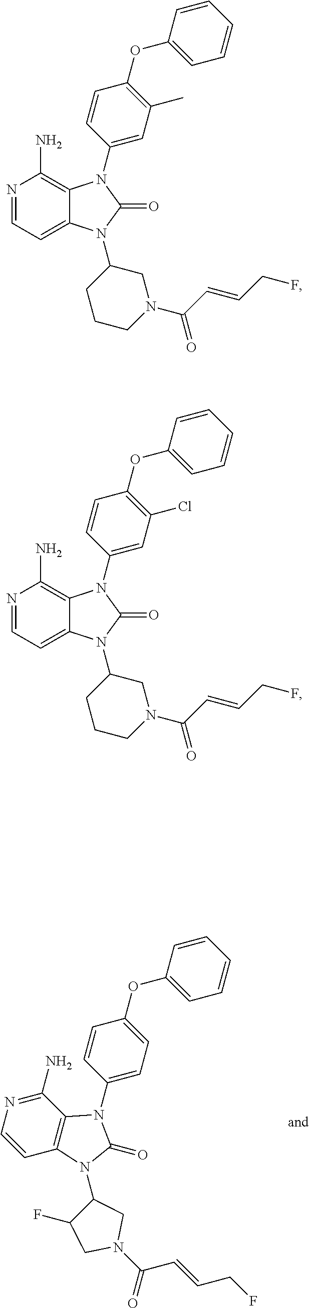 [SMILES: Cc1cc(-n2c(=O)n(C3CCCN(C(=O)/C=C/CF)C3)c3ccnc(N)c32)ccc1Oc1ccccc1 ; Nc1nccc2c1n(-c1ccc(Oc3ccccc3)c(Cl)c1)c(=O)n2C1CCCN(C(=O)/C=C/CF)C1 ; Nc1nccc2c1n(-c1ccc(Oc3ccccc3)cc1)c(=O)n2C1CN(C(=O)/C=C/CF)CC1F]

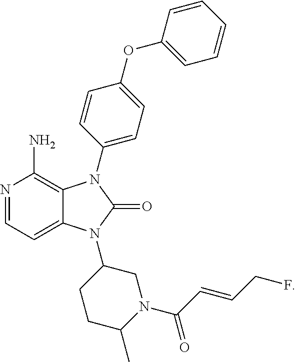 [SMILES: CC1CCC(n2c(=O)n(-c3ccc(Oc4ccccc4)cc3)c3c(N)nccc32)CN1C(=O)/C=C/CF]

22. The compound or the pharmaceutically acceptable salt thereof according to claim 21, the compound is selected from,

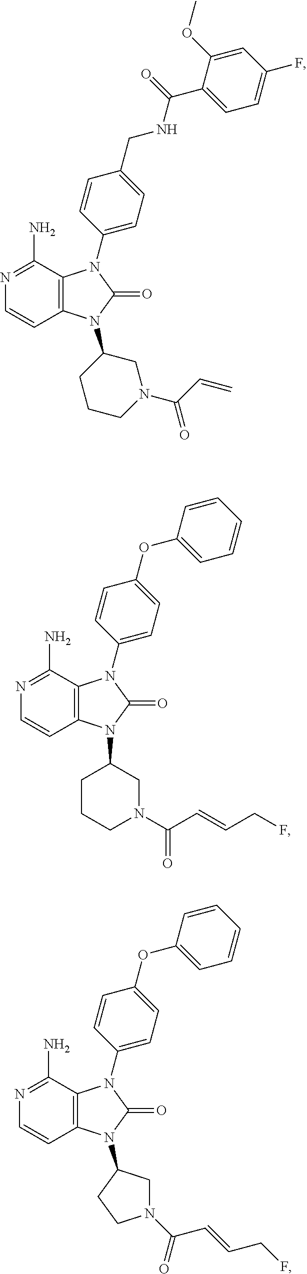 [SMILES: C=CC(=O)N1CCC[C@@H](n2c(=O)n(-c3ccc(CNC(=O)c4ccc(F)cc4OC)cc3)c3c(N)nccc32)C1 ; Nc1nccc2c1n(-c1ccc(Oc3ccccc3)cc1)c(=O)n2[C@@H]1CCCN(C(=O)/C=C/CF)C1 ; Nc1nccc2c1n(-c1ccc(Oc3ccccc3)cc1)c(=O)n2[C@@H]1CCN(C(=O)/C=C/CF)C1]

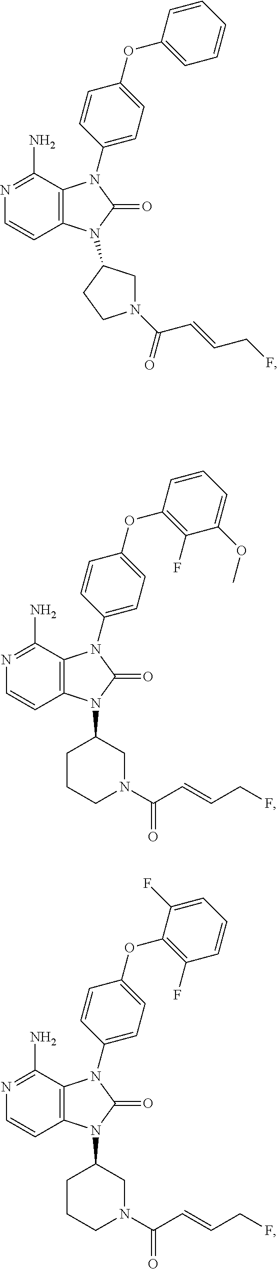 [SMILES: Nc1nccc2c1n(-c1ccc(Oc3ccccc3)cc1)c(=O)n2[C@H]1CCN(C(=O)/C=C/CF)C1 ; COc1cccc(Oc2ccc(-n3c(=O)n([C@@H]4CCCN(C(=O)/C=C/CF)C4)c4ccnc(N)c43)cc2)c1F ; Nc1nccc2c1n(-c1ccc(Oc3c(F)cccc3F)cc1)c(=O)n2[C@@H]1CCCN(C(=O)/C=C/CF)C1]

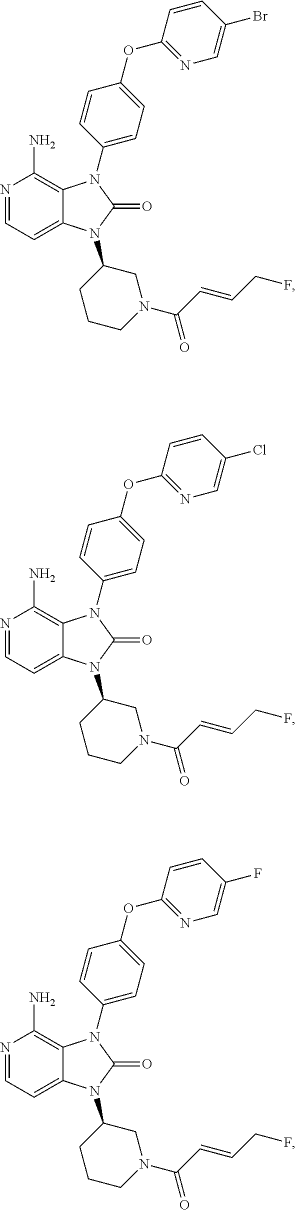 [SMILES: Nc1nccc2c1n(-c1ccc(Oc3ccc(Br)cn3)cc1)c(=O)n2[C@@H]1CCCN(C(=O)/C=C/CF)C1 ; Nc1nccc2c1n(-c1ccc(Oc3ccc(Cl)cn3)cc1)c(=O)n2[C@@H]1CCCN(C(=O)/C=C/CF)C1 ; Nc1nccc2c1n(-c1ccc(Oc3ccc(F)cn3)cc1)c(=O)n2[C@@H]1CCCN(C(=O)/C=C/CF)C1]

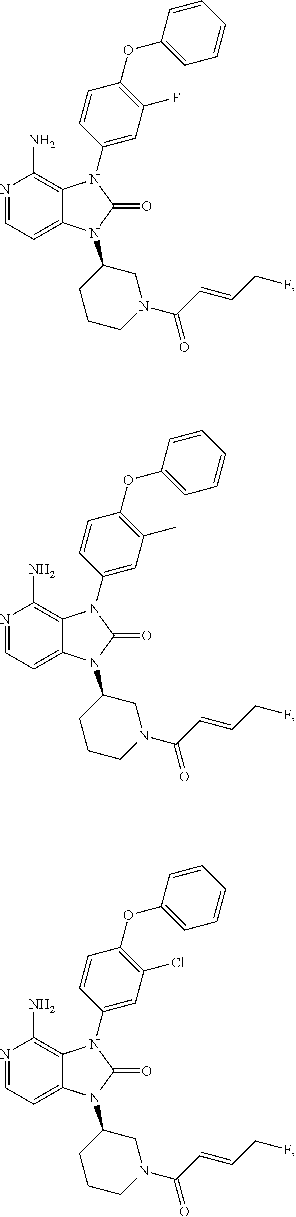 [SMILES: Nc1nccc2c1n(-c1ccc(Oc3ccccc3)c(F)c1)c(=O)n2[C@@H]1CCCN(C(=O)/C=C/CF)C1 ; Cc1cc(-n2c(=O)n([C@@H]3CCCN(C(=O)/C=C/CF)C3)c3ccnc(N)c32)ccc1Oc1ccccc1 ; Nc1nccc2c1n(-c1ccc(Oc3ccccc3)c(Cl)c1)c(=O)n2[C@@H]1CCCN(C(=O)/C=C/CF)C1]

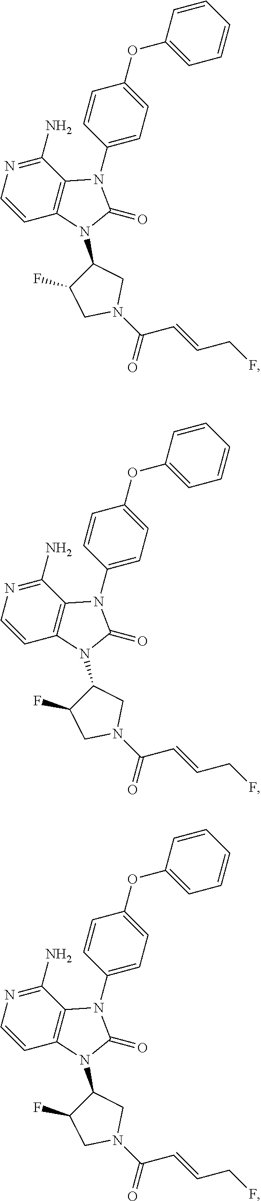 [SMILES: Nc1nccc2c1n(-c1ccc(Oc3ccccc3)cc1)c(=O)n2[C@H]1CN(C(=O)/C=C/CF)C[C@@H]1F ; Nc1nccc2c1n(-c1ccc(Oc3ccccc3)cc1)c(=O)n2[C@@H]1CN(C(=O)/C=C/CF)C[C@H]1F ; Nc1nccc2c1n(-c1ccc(Oc3ccccc3)cc1)c(=O)n2[C@H]1CN(C(=O)/C=C/CF)C[C@H]1F]

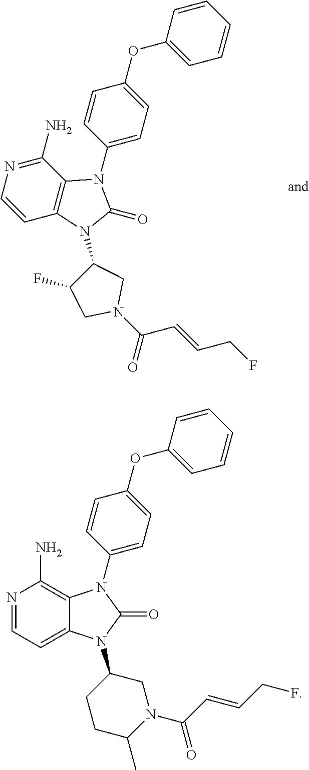 [SMILES: Nc1nccc2c1n(-c1ccc(Oc3ccccc3)cc1)c(=O)n2[C@@H]1CN(C(=O)/C=C/CF)C[C@@H]1F ; CC1CC[C@@H](n2c(=O)n(-c3ccc(Oc4ccccc4)cc3)c3c(N)nccc32)CN1C(=O)/C=C/CF]

23. A method for treating proliferation, inflammation, autoimmunity and other related disorders caused by protein kinases in a subject in need thereof, comprising: administering the compound or the pharmaceutically acceptable salt thereof according to claim 1 to the subject.

24. A method for treating autoimmune encephalomyelitis in a subject in need thereof, comprising: administering the compound or the pharmaceutically acceptable salt thereof according to claim 1 to the subject.
